(19) United States
(12) Patent Application Publication
(10) Pub. No.: US 2007/0265269 A1
(43) Pub. Date: Nov. 15, 2007

(54) Water Soluble Esters of [N-(4-Amino-2-Butynyl)] with Anticancer Activity

(76) Inventor: Zoser B. Salama, Berlin (DE)

Correspondence Address: JOYCE VON NATZMER;PEQUIGNOT + MYERS LLC, 200 Madison Avenue, Suite 1901, New York, NY, 10016, US

(21) Appl. No.: 10/598,563

(22) PCT Filed: Mar. 2, 2004
(86) PCT No.: PCT/EP04/02090

§ 371 Date: Jun. 5, 2007

(51) Int. Cl. A61K 31/5375; A61K 31/195; A61P 35/00; C07D 295/027; C07C 229/00; A61K 31/495

(52) U.S. Cl. 514/239.2; 514/252.12; 514/561; 544/171; 544/398; 560/155

(57) ABSTRACT

The present invention relates to water soluble esters of [N-(4-amino-2-butynyl)] or 4-(N-substituted amino)-2-butynyl-1-esters and methods for production of said esters and the use of the esters for treatment of cancer.

The present invention relates to water soluble esters of [N-(4-amino-2-butynyl)] or 4-(N-substituted amino)-2-butynyl-1-esters and methods for production of said esters and the use of the esters for treatment of cancer.

Cancer is a significant health problem in the world. Although advances have been made in cancer detection and treatment, no vaccine or other universally successful preventive or therapeutic method is currently available. Management of the disease currently relies on a combination of early diagnosis and aggressive treatment, which may include one or more of a variety of therapies such as surgery, radiotherapy, chemotherapy and hormone therapy. While such therapies provide benefit to many patients, a high mortality continues to be observed for many cancers. The development of improved anti-tumor agents would facilitate cancer prevention and treatment.

Unfortunately, cancer is the leading cause of death, second only to heart disease, of both men and women. In the fight against cancer, numerous techniques have been developed and are the subject of current research directed to understanding the nature and cause of the disease and to providing methods for the control or cure thereof.

Although thousands of potential anticancer agents have been evaluated, the treatment of human cancer remains fraught with complications which often present an array of suboptimnal treatment choices. As such, chemotherapeutic agents which possess little or no toxicity, which are inexpensive to obtain or manufacture, which are well tolerated by the patient, and which are easily administered would be a desirable addition to the therapeutic modalities currently available to the oncologist. Agents that will selectively sensitize malignant tissue to allow lower doses of radiation or therapy to achieve the same therapeutic effect with less damage to healthy tissues are also desirable. Similarly, agents that prevent cancer from occurring or reoccurring are also desirable. The present invention remedies these needs by providing such chemotherapeutic and sensitizing agents.

Therefore, the technical problem underlying the present invention is to provide alternative or further compounds with anticancer activity and methods for their production.

This problem is solved by the provision of the embodiments as defined in the claims.

It has been surprisingly discovered that water soluble esters of [N-(4-amino-2-butynyl)] or 4-(N-substituted amino)-2-butynyl-1-esters are useful in treatment of cancer. The present invention therefore relates to esters of [N-(4-amino-2-butynyl)] or 4-(N-substituted amino)-2-butynyl-1-esters and a pharmaceutical composition for use in therapy comprising the esters of [N-(4-amino-2-butynyl)] or 4-(N-substituted amino)-2-butynyl-1-esters.

The invention provides 4-(N-substituted amino)-2-butynyl-1-esters represented by the following general formula I their bis-2-(butynyl)diesters and pharmaceutically acceptable salts thereof, 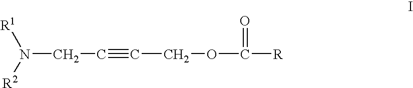
wherein
R is a hydrogen atom; a straight-chained or branched, saturated or unsaturated aliphatic radical with 1-20 C-atoms which is unsubstituted or substituted one or more times by C₁-C₆-alkyl, C₁-C₆-alkoxy, halogen, epoxy, amino, mercapto, a phenyl ring which is unsubstituted or substituted one or more times by C₁-C₆-alkyl, C₁-C₆-alkoxy, hydroxy, epoxy, amino, mercapto or halogen; a cycloalkyl group with 4 to 7 atoms unsubstituted or substituted one or more times by C₁-C₆-alkyl, C₁-C₆-alkoxy, hydroxy, epoxy, amino, mercapto or halogen,
R¹ and R² are joined to form a heterocyclic ring with 3 to 6 C-atoms, unsubstituted or substituted one or more times by C₁-C₆-alkyl, C₁-C₆-alkoxy, hydroxy, halogen, epoxy, amino, mercapto, whereby at least one C-atom can be replaced by O, S or N, or
R¹ and R₂ are the same or different a hydrogen atom, a straight-chained or branched, saturated or unsaturated aliphatic radical with 1-20 C-atoms, unsubstituted or substituted one or more times by C₁-C₆-alkyl, C₁-C₆-alkoxy, hydroxy, halogen, epoxy, amino, mercapto.

In a preferred embodiment the invention relates to said 4-(N-substituted amino)-2-butynyl-1-esters according, wherein R is a hydrogen atom, a straight-chained or branched alkyl group with 1-12 C-atoms, which can be substituted one or more times by C₁-C₆-alkyl; a phenyl ring which can be substituted one or more times by C₁-C₆-alkyl; a cyclo alkyl ring with 5-6 C-atoms which can be substituted one or more times by C₁-C₆-alkyl.

In a further embodiment the invention relates to said 4-(N-substituted amino)-2-butynyl-1-esters, wherein
R¹ and R² are the same alkyl group with 1-12 C-atoms, which can be straight-chained or branched and substituted by C₁-C₆-alkyl, or
R¹ and R² are joined to form a heterocyclic ring with 4 to 6 C-atoms, whereby at least one C-atom can be replaced by O, S or N, and the ring can be substituted by C₁-C₆-alkyl.

In another embodiment said 4-(N-substituted amino)-2-butynyl-1-esters is R a hydrogen atom, a straight-chained or branched alkyl group with 1-6 C-atoms, which can be substituted one or more times by C₁-C₆-alkyl; a phenyl ring which can be substituted one or more times by C₁-C₆-alkyl; a cyclo alkyl ring with 5-6 C-atoms which can be substituted one or more times by C₁-C₆-alkyl, and R¹ and R² are the same alkyl group with 1-6 C-atoms, which can be straight-chained or branched and substituted by C₁-C₆-alkyl, or R¹ and R² are joined to form a heterocyclic ring with 4 to 6 C-atoms, whereby at least one C-atom can be replaced by O, S or N, and the ring can be substituted by C₁-C₆-alkyl.

In a further embodiment the invention relates to said 4-(N-substituted amino)-2-butynyl-1-esters, wherein R is H or alkyl such as methyl, ethyl, propyl, butyl, pentyl, hexyl, phenyl, tertiary butyl and cyclohexyl and R¹ and R² are identically methyl, ethyl, propyl, butyl or phenyl; or form together with the N-atom a piperidino, pyrrolidino, morpholino, thiomorpholino, hexamethylene imino, piperazino and methyl piperazino ring.

In another embodiment of the invention, the 4-(N-substituted amino)-2-butynyl-1-esters are selected from the group comprising
[N-(4-morpholino-2-butynyl)]acetate
[N-(4-piperidino-2-butynyl)]acetate
[N-(4-(N-methyl piperazino-2-butynyl)]acetate
[N-(4-thiomorpholino-2-butynyl)]acetate
[N-(4-pyrrolidino-2-butynyl)]acetate
[N-(4-hexamethylene imino-2-butynyl)]acetate
[N-(4-morpholino-2-butynyl)]benzoate
[N-(4-morpholino-2-butynyl)]formate
[N-(4-diethylamino-2-butynyl)]acetate
[N-(4-diphenylamino-2-butynyl)]acetate
[N-(4-morpholino-2-butynyl)]propionate
[N-(4-thiomorpholino-2-butynyl)]propionate
[N-(4-morpholino-2-butynyl)]pivalate
[N,N′-(4,4-piperazino-bis-2-butynyl)]diacetate
[N-(4-morpholino-2-butynyl)]cyclohexyl carboxy late.

The compounds of this invention are useful in treating cancer. They are effective in inhibiting survival and/or growth of cancer cells and/or for inhibiting undesirable cell growth in general.

This invention further provides pharmaceutical and therapeutic compositions which contain a pharmaceutically or therapeutically effective amount of these conjugates and therapeutic methods and methods of treatment employing such methods. In particular, this invention relates to methods of treating cancer by administration of the 4-(N-substituted amino)-2-butynyl-1-esters disclosed herein. A method of treatment of cancer when multidrug resistance has occurred by administration of the conjugates and compositions containing such conjugates is also provided.

Accordingly, the present invention provides a method for production of esters of [N-(4-amino-2-butynyl)] or 4-(N-substituted amino)-2-butynyl-1-esters having the ability to inhibit abnormal cell growth in particular, to inhibit tumor cell growth and inhibit angeogenesis and the vascularization of endothelial cells.

The method for producing said 4-(N-substituted amino)-2-butynyl-1-esters or a pharmaceutically acceptable salt comprising a successive conversion of a propargyl alcohol in a propargyl ester by simple esterfication and a conversion of the propargyl ester in N-(4-amino-2-butynyl)ester by Mannich condensation. In a preferred method the Mannich condensation is performed in the presence of paraformaldehyd, an acid catalyst, Cu-salts (e.g. CuCl₂) and a solvent.

Also claimed is a kit for inhibit abnormal cell growth comprising the esters of [N-(4-amino-2-butynyl)] or 4-(N-substituted amino)-2-butynyl-1-esters, and/or synthetic analogues, modifications and pharmacologically active fragments thereof and an information about the using of parts of the kit.

Before the present compositions, formulations, and methods are described, it is to be understood that this invention is not limited to the particular methods, compositions, and cell lines described herein, as such methods, compositions, and cell lines may, of course, vary. It is also to be understood that the terminology used herein is for the purpose of describing particular embodiments only, and is not intended to limit the scope of the present invention which is only defined by the appended claims.

As used herein, including the appended claims, singular forms of words such as a, “an,” and “the” include their corresponding plural referents unless the context clearly dictates otherwise. Thus, e.g., reference to “an organism” includes one or more different organisms, reference to “a cell” includes one or more of such cells, and reference to “a method” includes reference to equivalent steps and methods known to a person of ordinary skill in the art, and so forth.

Unless otherwise defined, all technical and scientific terms used herein have the same meaning as commonly understood by a person of ordinary skill in the art to which this invention belongs. Although methods and materials similar or equivalent to those described herein can be used in the practice or testing of the present invention, suitable methods and materials are described below. All publications, patent applications, patents, and other references discussed above are provided solely for their disclosure prior to the filing date of the present application. Nothing herein is to be construed as an admission that the invention is not entitled to antedate such disclosure by virtue of its prior invention. All publications, patent applications, patents, and other references mentioned herein are incorporated by reference in their entirety including all figures and drawings.

Prior to setting forth the invention it may be helpful to an understanding thereof to set forth definitions of certain terms to be used hereinafter.

A “patient” for the purposes of the present invention includes both humans and other animals, particularly mammals, and other organisms. Thus the methods are applicable to both human therapy and veterinary applications. In the preferred embodiment the patient is a mammal, and in the most preferred embodiment the patient is human.

The term “animal” refers to an organism with a closed circulatory system of blood vessels and includes birds, mammals and crocodiles. The term “animal” used here also includes human subjects.

The term “angiogenesis” refers to the generation of new blood vessels into cells, tissue, organs or tumors.

The term “metastasis” refers to the process by which tumor cells are spread to distant parts of the body. The term is also used herein to refer to a tumor that develops through the metastatic process.

The term “contacting” is used herein interchangeably with the following: combined with, added to, mixed with, passed over, incubated with, flowed over, etc. Moreover, the compounds of present invention can be “administered” by any conventional method such as, for example, parenteral, oral, topical and inhalation routes as described herein.

As used herein, the term “safe and effective amount” refers to the quantity of a component that is sufficient to yield a desired therapeutic response without undue adverse side effects (such as toxicity, irritation, or allergic response) commensurate with a reasonable benefit/risk ratio when used in the manner of this invention. By “therapeutically effective amount” is meant an amount of a compound of the present invention effective to yield the desired therapeutic response. For example, an amount effective to delay the growth of or to cause a cancer, either a sarcoma or lymphoma, to shrink or not metastasize. The specific safe and effective amount or therapeutically effective amount will vary with such factors as the particular condition being treated, the physical condition of the patient, the type of mammal being treated, the duration of the treatment, the nature of concurrent therapy (if any), and the specific formulations employed and the structure of the compounds or its derivatives.

“An anti-angeogenic” amount refer to an amount of a compound or composition effective to depress, suppress or inhibit angiogenesis or result in amelioration of symptoms associated with an angiogenic disease. The desired result can be either a subjective relief of a symptom(s) or an objectively identifiable improvement in the recipient of the dosage, a decrease in the vascularization of endothelial cells or a decrease in the rate of angiogenesis as noted by a clinician or other qualified observer.

The terms “treating cancer,” “therapy,” and the like refer generally to any improvement in the mammal having the cancer wherein the improvement can be ascribed to treatment with the compounds of the present invention. The improvement can be either subjective or objective. For example, if the mammal is human, the patient may note improved vigor or vitality or decreased pain as subjective symptoms of improvement or response to therapy. Alternatively, the clinician may notice decrease in tumor size or tumor burden based on physical exam, laboratory parameters, tumor markers or radiographic findings. Some laboratory signs that the clinician may observe for response to therapy include normalization of tests such as white blood cell count, red blood cell count, platelet count, erythrocyte sedimentation rate, and various enzyme levels. Additionally, the clinician may observe a decrease in a detectable tumor marker. Alternatively, other tests can be used to evaluate objective improvement such as sonograms, nuclear magnetic resonance testing and positron emissions testing.

“Inhibiting the growth of tumor cells” can be evaluated by any accepted method of measuring whether growth of the tumor cells has been slowed or diminished. This includes direct observation and indirect evaluation such as subjective symptoms or objective signs as discussed above.

Accordingly, the compositions of the invention are administered to cells. By “administered” herein is meant administration of a therapeutically effective dose of the candidate agents of the invention to a cell either in cell culture or in a patient. By “therapeutically effective dose” herein is meant a dose that produces the effects for which it is administered. The exact dose will depend on the purpose of the treatment, and will be ascertainable by one skilled in the art using known techniques. As is known in the art, adjustments for systemic versus localized delivery, age, body weight, general health, sex, diet, time of administration, drug interaction and the severity of the condition may be necessary, and will be ascertainable with routine experimentation by those skilled in the art. By “cells” herein is meant almost any cell in which mitosis or meiosis can be altered.

Therefore, the present invention relates to a pharmaceutical anti-cancer composition comprising a therapeutically effective amount of 4-(N-substituted amino)-2-butynyl-1-esters represented by the following general formula I, their bis-(2-butynyl)diesters and pharmaceutically acceptable salts thereof, 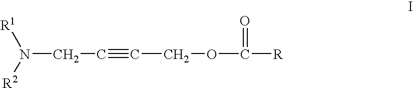
wherein
R is a hydrogen atom; a straight-chained or branched, saturated or unsaturated aliphatic radical with 1-20 C-atoms which is unsubstituted or substituted one or more times by C₁-C₆-alkyl, C₁-C₆-alkoxy, hydroxy, halogen, epoxy, amino, mercapto, a phenyl ring which is unsubstituted or substituted one or more times by C₁-C₆-alkyl, C₁-C₆-alkoxy, hydroxy, epoxy, amino, mercapto or halogen; a cycloalkyl group with 4 to 7 atoms unsubstituted or substituted one or more times by C₁-C₆-alkyl, C₁-C₆-alkoxy, hydroxy, epoxy, amino, mercapto or halogen,
R¹ and R² are joined to form a heterocyclic ring with 3 to 6 C-atoms, unsubstituted or substituted one or more times by C₁-C₆-alkyl, C₁-C₆-alkoxy, hydroxy, halogen, epoxy, amino, mercapto, whereby at least one C-atom can be replaced by O, S or N, or
R¹ and R² are the same or different a hydrogen atom, a straight-chained or branched, saturated or unsaturated aliphatic radical with 1-20 C-atoms, unsubstituted or substituted one or more times by C₁-C₆-alkyl, C₁-C₆-alkoxy, hydroxy, halogen, epoxy, amino, mercapto,
   for manufacturing an agent for the treatment of a cell proliferative disorder and one or more pharmaceutically acceptable adjuvant, excipient, carrier, buffer, diluent and/or customary pharmaceutical auxiliary. In a preferred embodiment of the invention the ester of the invention can be administered in a pharmaceutically acceptable formulation. The present invention pertains to any pharmaceutically acceptable formulations, such as synthetic or natural polymers in the form of macromolecular complexes, nanocapsules, microspheres, or beads, and lipid-based formulations including oil-in-water emulsions, micelles, mixed micelles, synthetic membrane vesicles, and resealed erythrocytes. In addition to the ester and the pharmaceutically acceptable polymer, the pharmaceutically acceptable formulation used in the method of the invention can comprise additional pharmaceutically acceptable carriers and/or excipients. As used herein, pharmaceutically acceptable carrier includes any and all solvents, dispersion media, coatings, antibacterial and anti fungal agents, isotonic and absorption delaying agents, and the like that are physiologically compatible. For example, the carrier can be suitable for injection into the blood. Excipients include pharmaceutically acceptable stabilizers and disintegrants. In another embodiment, the pharmaceutically acceptable formulations comprise lipid-based formulations. Any of the known lipid-based drug delivery systems can be used in the practice of the invention. For instance, multivesicular liposomes (MVL), multilamellar liposomes (also known as multilamellar vesicles or MLV), unilamellar liposomes, including small unilamellar liposomes (also known as unilamellar vesicles or SUV) and large unilamellar liposomes (also known as large unilamellar vesicles or LUV), can all be used so long as a sustained release rate of the encapsulated esters can be established. In one embodiment, the lipid-based formulation can be a multivesicular liposome system. The composition of the synthetic membrane vesicle is usually a combination of phospholipids, usually in combination with steroids, especially cholesterol. Other phospholipids or other lipids may also be used. Examples of lipids useful in synthetic membrane vesicle production include phosphatidylglycerols, phosphatidylcholines, phosphatidyl-serines, phosphatidylethanolaminos, sphingolipids, cerebrosides, and gangliosides. Preferably phospholipids including egg phosphatidylcholine, dipalmitoylphosphatidylcholine, distearoylphosphatidylcholine, dioleoylphosphatidylcholine, dipalmitoylphosphatidylglycerol, and dioleoylphosphatidylglycerol are used. In another embodiment, the composition containing the ester may be incorporated or impregnated into a bioabsorbable matrix. In addition, the matrix may be comprised of the said biopolymer. A suitable biopolymer for the present invention can include also one or more macromolecules selected from the group consisting of collagen, elastin, fibronectin, vitronectin, laminin, polyglycolic acid, hyaluronic acid, chondroitin sulfate, dermatan sulfate, heparin sulfate, heparin, fibrin, cellulose, gelatin, polylysine, echinonectin, entactin, thrombospondin, uvomorulin, biglycan, decorin, and dextran. The formulation of these macromolecules into a biopolymer is well known in the art. In a preferred embodiment, the therapeutic composition is not immunogenic when administered to a human patient for therapeutic purposes.

A therapeutic composition of the present invention can include pharmaceutically acceptable salts of the components therein. Pharmaceutically acceptable salts include the acid addition salts that are formed with inorganic acids such as, for example, hydrochloric or phosphoric acids, or such organic acids as acetic, tartaric, mandelic and the like. Salts formed with the free carboxyl groups can also be derived from inorganic bases such as, for example, sodium, potassium, ammonium, calcium or ferric hydroxides, and such organic bases as isopropylamine, trimethylamino, 2-ethylamino ethanol, histidine, procaine and the like. Physiologically tolerable carriers are well known in the art. Exemplary of liquid carriers are sterile aqueous solutions that contain no materials in addition to the active ingredients and water, or contain a buffer such as sodium phosphate at physiological pH value, physiological saline or both, such as phosphate-buffered saline. Still further, aqueous carriers can contain more than one buffer salt, as well as salts such as sodium and potassium chlorides, dextrose, propylene glycol, polyethylene glycol and other solutes. Liquid compositions can also contain liquid phases in addition to and to the exclusion of water. Exemplary of such additional liquid phases are glycerin, vegetable oils such as cottonseed oil, organic esters such as ethyl oleate, and water-oil emulsions. A therapeutic composition contains a polypeptide of the present invention, typically an amount of at least 0.1 weight percent of polypeptide per weight of total therapeutic composition. A weight percent is a ratio by weight of polypeptide to total composition. Thus, for example, 0.1 weight percent is 0.1 grams of polypeptide per 100 grams of total composition.

The term “pharmaceutically acceptable salt” refers to those salts of compounds which retain the biological effectiveness and properties of the free bases and which are obtained by reaction with inorganic acids such as hydrochloric acid, hydrobromic acid, sulfuric acid, nitric acid, phosphoric acid, methanesulfonic acid, ethanesulfonic acid, ptoluenesulfonic acid, salicylic acid and the like. Pharmaceutically acceptable salts include, for example, alkali metal salts, such as sodium and potassium, alkaline earth salts and ammonium salts.

The pharmaceutical composition containing the active ingredient may be in a form suitable for oral use, for example, as tablets, troches, lozenges, aqueous or oily suspensions, dispersible powders or granules, emulsions, hard or soft capsules, or syrups or elixirs. Compositions intended for oral use may be prepared according to any method known to the art for the manufacture of pharmaceutical compositions and such compositions may contain one or more agents selected from the group consisting of sweetening agents, flavouring agents, colouring agents and preserving agents in order to provide pharmaceutically elegant and palatable preparations. Tablets contain the active ingredient in admixture with non-toxic pharmaceutically acceptable excipients which are suitable for the manufacture of tablets. These excipients may be for example, inert diluents, such as calcium carbonate, sodium carbonate, lactose, calcium phosphate or sodium phosphate; granulating and disintegrating agents, for example corn starch, or alginic acid; binding agents, for example starch, gelatin or acacia, and lubricating agents, for example magnesium stearate, stearic acid or talc. The tablets may be uncoated or they may be coated by known techniques to delay disintegration and absorption in the gastointestinal tract and thereby provide a sustained action over a longer period. For example, a time delay material such as glyceryl monostearate or glyceryl distearate may be employed. They may also be coated by the techniques described in the U.S. Pat. Nos. 4,256,108; 4,166,452; and 4,265,874, to form osmotic therapeutic tablets for control release. A pharmaceutical composition may also, or alternatively, contain one or more drugs, which may be linked to a modulating agent or may be free within the composition. Virtually any drug may be administered in combination with a modulating agent as described herein, for a variety of purposes as described below. Examples of types of drugs that may be administered with a modulating agent include analgesics, anesthetics, antianginals, antifungals, antibiotics, anticancer drugs (e.g., taxol or mitomycin C), antiinflammatories (e.g., ibuprofen and indomethacin), anthelmintics, antidepressants, antidotes, antiemetics, antihistamines, antihypertensives, antimalarials, antimicrotubule agents (e.g., colchicine or vinca alkaloids), antimigraine agents, antimicrobials, antiphsychotics, antipyretics, antiseptics, anti-signaling agents (e.g., protein kinase C inhibitors or inhibitors of intracellular calcium mobilization), antiarthritics, antithrombin agents, antituberculotics, antitussives, antivirals, appetite suppressants, cardioactive drugs, chemical dependency drugs, cathartics, chemotherapeutic agents, coronary, cerebral or peripheral vasodilators, contraceptive agents, depressants, diuretics, expectorants, growth factors, hormonal agents, hypnotics, immunosuppression agents, narcotic antagonists, parasympathomimetics, sedatives, stimulants, sympathomimetics, toxins (e.g., cholera toxin), tranquilizers and urinary antiinfectives.

Formulations for oral use may also be presented as hard gelatin capsules where in the active ingredient is mixed with an inert solid diluent, for example calcium carbonate, calcium phosphate or kaolin, or as soft gelatin capsules wherein the active ingredient is mixed with water or an oil medium, for example peanut oil, liquid paraffin or olive oil.

Aqueous suspensions contain the active materials in admixture with excipients suitable for the manufacture of aqueous suspensions. Such excipients are suspending agents, for example sodium carboxymethylcellulose, methylcellulose, hydroxy-propylmethylcellulose, sodium alginate polyvinyl-pyrrolidone, gum tragacanth and gum acacia; dispersing or wetting agents may be a naturally occurring phosphatide, for example lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such a polyoxyethylene with partial esters derived from fatty acids and hexitol anhydrides, for example polyoxyethylene sorbitan monooleate. The aqueous suspensions may also contain one or more preservatives, for example ethyl, or n-propyl, p-hydroxybenzoate, one or more colouring agents, one or more flavouring agents, and one or more sweetening agents, such as sucrose or saccharin.

Oily suspensions may be formulated by suspending the active ingredient in a vegetable oil, for example arachis oil, olive oil, sesame oil or coconut oil, or in a mineral oil such as liquid paraffin. The oily suspensions may contain a thickening agent, for example beeswax, hard paraffin or cetyl alcohol. Sweetening agents such as those set forth above, and flavouring agents may be added to provide a palatable oral preparation. These compositions may be preserved by the addition of an anti-oxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified, for example sweetening, flavouring and colouring agents, may also be present.

The pharmaceutical compositions of the invention may also be in the form of oil-in-water emulsions. The oily phase may be a vegetable oil, for example olive oil or arachis oil, or a mineral oil, for example liquid paraffin or mixtures of these. Suitable emulsifying agents may be naturally-occurring gums, for example gum acacia or gum tragacanth, naturally-occurring phosphatides, for example soya bean, lecithin, and esters or partial esters derived from fatty acids and hexitol anhydrides, for example sorbitan monooleate and condensation products of the said partial esters with ethylene oxide, for example polyoxyethylene sorbitan monooleate. The emulsions may also contain sweetening and flavouring agents.

Syrups and elixirs may be formulated with sweetening agents, for example glycerol, propylene glycol, sorbitol or sucrose. Such formulations may also contain a demulcent, a preservative and flavouring and colouring agents. The pharmaceutical compositions may be in the form of a sterile injectable aqueous or oleagenous suspension. This suspension may be formulated according to the known art using those suitable dispersing or wetting agents and suspending agents which have been mentioned above. The sterile injectable preparation may also be in a sterile injectable solution or suspension in a non-toxic parenterally-acceptable diluent or solvent, for example as absolution in 1,3-butane diol. Among the acceptable vehicles and solvents that may be employed are water, Ringer's solution and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

Dosage levels of the order of from about 0.05 mg to about 140 mg per kilogram of body weight per: day are useful in the treatment of the above-indicated conditions (about 2.5 mg to about 7 g per patient per day). For example, inflammation may be effectively treated by the administration of from about 0.01 to 50 mg of the compound per kilogram of body weight per day (about 0.5 mg to about 3.5 g per patient per day). The amount of active ingredient that may be combined with the carrier materials to produce a single dosage form will vary depending upon the host treated and the particular mode of administration. For example, a formulation intended for the oral administration of humans may vary from about 5 to about 95% of the total composition. Dosage unit forms will generally contain between from about 1 mg to about 500 mg of active ingredient. It will be understood, however, that the specific dose level for any particular patient will depend upon a variety of factors including the activity of the specific compound employed, the age, body weight, general health, sex, diet time of administration, route of administration, rate of excretion, drug combination and the severity of the particular disease undergoing therapy. The dosage effective amount of compounds according to the invention will vary depending upon factors including the particular compound, toxicity, and inhibitory activity, the condition treated, and whether the compound is administered alone or with other therapies. Typically a dosage effective amount will range from about 0.0001 mg/kg to 1500 mg/kg, more preferably 1 to 1000 mg/kg, more preferably from about 1 to 150 mg/kg of body weight, and most preferably about 50 to 100 mg/kg of body weight. The invention relates also to a process or a method for the treatment of the abovementioned pathological conditions. The compounds of the present invention can be administered prophylactically or therapeutically, preferably in an amount that is effective against the mentioned disorders, to a warm-blooded animal, for example a human, requiring such treatment, the compounds preferably being used in the form of pharmaceutical compositions.

Formulation of pharmaceutically-acceptable excipients and carrier solutions is well-known to those of skill in the art, as is the development of suitable dosing and treatment regimens for using the particular compositions described herein in a variety of treatment regimens, including e.g., oral, parenteral, intravenous, intranasal, and intra-muscular administration and formulation.

A. Oral Delivery

In certain applications, the pharmaceutical compositions disclosed herein may be delivered via oral administration to an animal. As such, these compositions may be formulated with an inert diluent or with an assimilable edible carrier, or they may be enclosed in hard- or soft-shell gelatin capsule, or they may be compressed into tablets, or they may be incorporated directly with the food of the diet.

The active compounds may even be incorporated with excipients and used in the form of ingestible tablets, buccal tables, troches, capsules, elixirs, suspensions, syrups, wafers, and the like. The tablets, troches, pills, capsules and the like may also contain the following: a binder, as gum tragacanth, acacia, cornstarch, or gelatin; excipients, such as dicalcium phosphate; a disintegrating agent, such as corn starch, potato starch, alginic acid and the like; a lubricant, such as magnesium stearate; and a sweetening agent, such as sucrose, lactose or saccharin may be added or a flavoring agent, such as peppermint, oil of wintergreen, or cherry flavoring. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar, or both. A syrup of elixir may contain the active compound sucrose as a sweetening agent methyl and propylparabens as preservatives, a dye and flavoring, such as cherry or orange flavor. Of course, any material used in preparing any dosage unit form should be pharmaceutically pure and substantially non-toxic in the amounts employed. In addition, the active compounds may be incorporated into sustained-release preparation and formulations.

Typically, these formulations may contain at least about 0.1% of the active compound or more, although the percentage of the active ingredient(s) may, of course, be varied and may conveniently be between about 1 or 2% and about 60% or 70% or more of the weight or volume of the total formulation. Naturally, the amount of active compound(s) in each therapeutically useful composition may be prepared is such a way that a suitable dosage will be obtained in any given unit dose of the compound. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable.

For oral administration the compositions of the present invention may alternatively be incorporated with one or more excipients in the form of a mouthwash, dentifrice, buccal tablet, oral spray, or sublingual orally-administered formulation. For example, a mouthwash may be prepared incorporating the active ingredient in the required amount in an appropriate solvent, such as a sodium borate solution (Dobell's Solution). Alternatively, the active ingredient may be incorporated into an oral solution such as one containing sodium borate, glycerin and potassium bicarbonate, or dispersed in a dentifrice, or added in a therapeutically-effective amount to a composition that may include water, binders, abrasives, flavoring agents, foaming agents, and humectants. Alternatively the compositions may be fashioned into a tablet or solution form that may be placed under the tongue or otherwise dissolved in the mouth.

B. Injectable Delivery

In certain circumstances it will be desirable to deliver the pharmaceutical compositions disclosed herein parenterally, intravenously, intramuscularly, or even intraperitoneally. Solutions of the active compounds as free base or pharmacologically acceptable salts may be prepared in water suitably mixed with a surfactant, such as hydroxypropylcellulose. Dispersions may also be prepared in glycerol, liquid polyethylene glycols, and mixtures thereof and in oils. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases the form must be sterile and must be fluid to the extent that easy syringability exists. It must be stable under the conditions of manufacture and storage and must be preserved against the contaminating action of microorganisms, such as bacteria and fungi. The carrier can be a solvent- or dispersion medium containing, for example, water, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be facilitated by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, a sterile aqueous medium that can be employed will be known to those of skill in the art in light of the present disclosure. For example, one dosage may be dissolved in 1 ml of isotonic NaCl solution and either added to 1000 ml of hypodermoclysis fluid or injected at the proposed site of infusion. Some variation in dosage will necessarily occur depending on the condition of the subject being treated. The person responsible for administration will, in any event, determine the appropriate dose for the individual subject. Moreover, for human administration, preparations should meet sterility, pyrogenicity, and the general safety and purity standards as required by national or regional offices of biologics standards.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

The compositions disclosed herein may be formulated in a neutral or salt form. Pharmaceutically-acceptable salts, include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. Upon formulation, solutions will be administered in a manner compatible with the dosage formulation and in such amount as is therapeutically effective. The formulations are easily administered in a variety of dosage forms such as injectable solutions, drug-release capsules, and the like.

As used herein, “carrier” includes any and all solvents, dispersion media, vehicles, coatings, diluents, antibacterial and antifingal agents, isotonic and absorption delaying agents, buffers, carrier solutions, suspensions, colloids, and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions.

The phrase “pharmaceutically-acceptable” refers to molecular entities and compositions that do not produce an allergic or similar untoward reaction when administered to a human. The preparation of an aqueous composition that contains a protein as an active ingredient is well understood in the art. Typically, such compositions are prepared as injectables, either as liquid solutions or suspensions; solid forms suitable for solution in, or suspension in, liquid prior to injection can also be prepared. The preparation can also be emulsified.

c. Nasal Delivery

In certain embodiments, the pharmaceutical compositions may be delivered by intranasal sprays, inhalation, and/or other aerosol delivery vehicles. Likewise, the delivery of drugs using intranasal microparticle resins and lysophosphatidyl-glycerol compounds are also well-known in the pharmaceutical arts.

D. Liposome-, Nanocapsule-, and Microparticle-Mediated Delivery

In certain embodiments, the inventors contemplate the use of liposomes, nanocapsules, microparticles, microspheres, lipid particles, vesicles, and the like, for the introduction of the compositions of the present invention into suitable host cells. In particular, the compositions of the present invention may be formulated for delivery either encapsulated in a lipid particle, a liposome, a vesicle, a nanosphere, or a nanoparticle or the like.

Such formulations may be preferred for the introduction of pharmaceutically-acceptable formulations of the esters or constructs disclosed herein. The formation and use of liposomes is generally known to those of skill in the art. Recently, liposomes were developed with improved serum stability and circulation half-times (Gabizon and Papahadjopoulos, 1988). Further, various methods of liposome and liposome like preparations as potential drug carriers have been reviewed (Takakura, 1998; Chandran et al., 1997; Margalit, 1995; U.S. Pat. Nos. 5,567,434; 5,552,157; 5,565,213; 5,738,868 and 5,795,587, each specifically incorporated herein by reference in its entirety). In addition to liposome characteristics, an important determinant in entrapping compounds is the physicochemical properties of the compound itself. Polar compounds are trapped in the aqueous spaces and nonpolar compounds bind to the lipid bilayer of the vesicle. Polar compounds are released through permeation or when the bilayer is broken, but nonpolar compounds remain affiliated with the bilayer unless it is disrupted by temperature or exposure to lipoproteins. Both types show maximum efflux rates at the phase transition temperature. Liposomes interact with cells via four different mechanisms: endocytosis by phagocytic cells of the reticuloendothelial system such as macrophages and neutrophils; adsorption to the cell surface, either by nonspecific weak hydrophobic or electrostatic forces, or by specific interactions with cell-surface components; fusion with the plasma cell membrane by insertion of the lipid bilayer of the liposome into the plasma membrane, with simultaneous release of liposomal contents into the cytoplasm; and by transfer of liposomal lipids to cellular or subcellular membranes, or vice versa, without any association of the liposome contents. It often is difficult to determine which mechanism is operative and more than one may operate at the same time.

The fate and disposition of intravenously injected liposomes depend on their physical properties, such as size, fluidity, and surface charge. They may persist in tissues for h or days, depending on their composition, and half lives in the blood range from min to several h. Larger liposomes, such as MLVs and LUVs, are taken up rapidly by phagocytic cells of the reticuloendothelial system, but physiology of the circulatory system restrains the exit of such large species at most sites. They can exit only in places where large openings or pores exist in the capillary endothelium, such as the sinusoids of the liver or spleen. Thus, these organs are the predominate site of uptake. On the other hand, SUVs show a broader tissue distribution but still are sequestered highly in the liver and spleen. In general, this in vivo behavior limits the potential targeting of liposomes to only those organs and tissues accessible to their large size. These include the blood, liver, spleen, bone marrow, and lymphoid organs.

Targeting is generally not a limitation in terms of the present invention. However, should specific targeting be desired, methods are available for this to be accomplished. Antibodies may be used to bind to the liposome surface and to direct the antibody and its drug contents to specific antigenic receptors located on a particular cell-type surface. Carbohydrate determinants (glycoprotein or glycolipid cell-surface components that play a role in cell-cell recognition, interaction and adhesion) may also be used as recognition sites as they have potential in directing liposomes to particular cell types. Mostly, it is contemplated that intravenous injection of liposomal preparations would be used, but other routes of administration are also conceivable.

Alternatively, the invention provides for pharmaceutically-acceptable nanocapsule formulations of the compositions of the present invention. Nanocapsules can generally entrap compounds in a stable and reproducible way (Henry-Michelland et al., 1987; Quintanar-Guerrero et al., 1998; Douglas et al., 1987). To avoid side effects due to intracellular polymeric overloading, such ultrafine particles (sized around 0.1 .mu.m) should be designed using polymers able to be degraded in vivo. Biodegradable polyalkyl-cyanoacrylate nanoparticles that meet these requirements are contemplated for use in the present invention.

The subjects treated will typically comprise mammals and most preferably will be human subjects, e.g., human cancer subjects. The compounds of the invention may be used alone or in combination. Additionally, the treated compounds may be utilized with other types of treatments, e.g., cancer treatments. For example, the subject compounds may be used with other chemotherapies, e.g., tamoxifen, taxol, methothrexate, biologicals, such as antibodies, growth factors, lymphokines, or radiation, etc. Combination therapies may result in synergistic results. The preferred indication is cancer, especially the cancers identified previously.

The compositions and methods provided herein are particularly deemed useful for the treatment of cancer including solid tumors such as skin, breast, brain, cervical carcinomas, testicular carcinomas, etc. More particularly, cancers that may be treated by the compositions and methods of the invention include, but are not limited to: Cardiac: sarcoma (angiosarcoma, fibrosarcoma, rhabdomyosarcoma, liposarcoma), myxoma, rhabdomyoma, fibroma, lipoma and teratoma; Lung: bronchogenic carcinoma (squamous cell, undifferentiated small cell, undifferentiated large cell, adenocarcinoma), alveolar (bronchiolar) carcinoma, bronchial adenoma, sarcoma, lymphoma, chondromatous hamartoma, mesotheliorna; Gastrointestinal: esophagus (squamous cell carcinoma, adenocarcinoma, leiomyosarcoma, lymphoma), stomach (carcinoma, lymphoma, leiomyosarcoma), pancreas (ductal adenocarcinoma, insulinoma, glucagonoma, gastrinoma, carcinoid tumors, vipoma), small bowel (adenocarcinoma, lymphoma, carcinoid tumors, Karposi's sarcoma, leiomyoma, hemangioma, lipoma, neurofibroma, fibroma), large bowel (adenocarcinoma, tubular adenoma, villous adenoma, hamartoma, leiomyoma); Genitourinary tract: kidney (adenocarcinoma, Wilm's tumor [nephroblastoma], lymphoma, leukemia), bladder and urethra (squamous cell carcinoma, transitional cell carcinoma, adenocarcinoma), prostate (adenocarcinoma, sarcoma), testis (seminoma, teratoma, embryonal carcinoma, teratocarcinoma, choriocarcinoma, sarcoma, interstitial cell carcinoma, fibroma, fibroadenoma, adenomatoid tumors, lipoma); Liver: hepatoma (hepatocellular carcinoma), cholangiocarcinoma, hepatoblastoma, angiosarcoma, hepatocellular adenoma, hemangioma; Bone: osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochronfroma (osteocartilaginous exostoses), benign chondroma, chondroblastoma, chondromyxofibroma, osteoid osteoma and giant cell tumors; Nervous system: skull (osteoma, hemangioma, granuloma, xanthoma, osteitis deformans), meninges (meningioma, meningiosarcoma, gliomatosis), brain (astrocytoma, medulloblastoma, glioma, ependymoma, germinoma [pinealoma], glioblastoma multiform, oligodendroglioma, schwannoma, retinoblastoma, congenital tumors), spinal cord neurofibroma, meningioma, glioma, sarcoma); Gynecological: uterus (endometrial carcinoma), cervix (cervical carcinoma, pre-tumor cervical dysplasia), ovaries (ovarian carcinoma [serous cystadenocarcinorna, mucinous cystadenocarcinoma, unclassified carcinoma], granulosa-thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma], fallopian tubes (carcinoma); Hematologic: blood (myeloid leukemia [acute and chronic], acute lymphoblastic leukemia, chronic lymphocytic leukemia, myeloproliferative diseases, multiple myeloma, myelodysplastic syndrome), Hodgkin's disease, non-Hodgkin's lymphoma [malignant lymphoma]; Skin: malignant melanoma, basal cell carcinoma, squamous cell carcinoma, Karposi's sarcoma, moles dysplastic nevi, lipoma, angioma, dermatofibroma, keloids, psoriasis; and Adrenal glands: neuroblastoma. Thus, the term “cancerous cell” as provided herein, includes a cell afflicted by any one of the above identified conditions.

Therefore, as used herein, “cancer” refers to all types of cancer or neoplasm or malignant tumors found in mammals, including carcinomas and sarcomas. Examples of cancers are cancer of the brain, breast, cervix, colon, head & neck, kidney, lung, non-small cell lung, melanoma, mesothelioma, ovary, sarcoma, stomach, uterus and Medulloblastoma.

The term “leukemia” refers broadly to progressive, malignant diseases of the blood-forming organs and is generally characterized by a distorted proliferation and development of leukocytes and their precursors in the blood and bone marrow. Leukemia is generally clinically, classified on the basis of (1) the duration and character of the disease-acute or chronic; (2) the type of cell involved; myeloid (myelogenous), lymphoid (lymphogenous), or monocytic; and (3) the increase or non-increase in the number abnormal cells in the blood-leukemic or aleukemic (subleukemic). The P388 leukemia model is widely accepted as being predictive of in vivo anti-leukemic activity. It is believed that compound that tests positive in the P388 assay will generally exhibit some level of anti-leukemic activity in vivo regardless of the type of leukemia being treated. Accordingly, the present invention includes a method of treating leukemia, and, preferably, a method of treating acute nonlymphocytic leukemia, chronic lymphocytic leukemia, acute granulocytic leukemia, chronic granulocytic leukemia, acute promyelocytic leukemia, adult T-cell leukemia, aleukemic leukemia, a leukocythemic leukemia, basophylic leukemia, blast cell leukemia, bovine leukemia, chronic myelocytic leukemia, leukemia cutis, embryonal leukemia, eosinophilic leukemia, Gross' leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocytic leukemia, micromyeloblastic leukemia, monocytic leukemia, myeloblastic leukemia, myelocytic leukemia, myeloid granulocytic leukemia, myelomonocytic leukemia, Naegeli leukemia, plasma cell leukemia, plasmacytic leukemia, promyelocytic leukemia, Rieder cell leukemia, Schilling's leukemia, stem cell leukemia, subleukemic leukemia, and undifferentiated cell leukemia.

The term “sarcoma” generally refers to a tumor which is made up of a substance like the embryonic connective tissue and is generally composed of closely packed cells embedded in a fibrillar or homogeneous substance. Sarcomas which can be treated with ester of the invention and optionally a potentiator and/or chemotherapeutic agent include a chondrosarcoma, fibrosarcoma, lymphosarcoma, melanosarcoma, myxosarcoma, osteosarcoma, Abemethy's sarcoma, adipose sarcoma, liposarcoma, alveolar soft part sarcoma, ameloblastic sarcoma, botryoid sarcoma, chloroma sarcoma, chorio carcinoma, embryonal sarcoma, Wilms' tumor sarcoma, endometrial sarcoma, stromal sarcoma, Ewing's sarcoma, fascial sarcoma, fibroblastic sarcoma, giant cell sarcoma, granulocytic sarcoma, Hodgkin's sarcoma, idiopathic multiple pigmented hemorrhagic sarcoma, immunoblastic sarcoma of B cells, lymphoma, immunoblastic sarcoma of T-cells, Jensen's sarcoma, Kaposi's sarcoma, Kupffer cell sarcoma, angiosarcoma, leukosarcoma, malignant mesenchymoma sarcoma, parosteal sarcoma, reticulocytic sarcoma, Rous sarcoma, serocystic sarcoma, synovial sarcoma, and telangiectaltic sarcoma.

The term “melanoma” is taken to mean a tumor arising from the melanocytic system of the skin and other organs. Melanomas which can be treated with said esters and optionally a potentiator and/or another chemotherapeutic agent include, for example, acral-lentiginous melanoma, amelanotic melanoma, benign juvenile melanoma, Cloudman's melanoma, S91 melanoma, Harding-Passey melanoma, juvenile melanoma, lentigo maligna melanoma, malignant melanoma, nodular melanoma, subungal melanoma, and superficial spreading melanoma.

The term “carcinoma” refers to a malignant new growth made up of epithelial cells tending to infiltrate the surrounding tissues and give rise to metastases. Exemplary carcinomas which can be treated with said ester and optionally a potentiator and/or a chemotherapeutic agent include, for example, acinar carcinoma, acinous carcinoma, adenocystic carcinoma, adenoid cystic carcinoma, carcinoma adenomatosum, carcinoma of adrenal cortex, alveolar carcinoma, alveolar cell carcinoma, basal cell carcinoma, carcinoma basocellulare, basaloid carcinoma, basosquamous cell carcinoma, bronchioalveolar carcinoma, bronchiolar carcinoma, bronchogenic carcinoma, cerebriform carcinoma, cholangiocellular carcinoma, chorionic carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epiermoid carcinoma, carcinoma epitheliale adenoides, exophytic carcinoma, carcinoma ex ulcere, carcinoma fibrosum, gelatiniformi carcinoma, gelatinous carcinoma, giant cell carcinoma, carcinoma gigantocellulare, glandular carcinoma, granulosa cell carcinoma, hair-matrix carcinoma, hematoid carcinoma, hepatocellular carcinoma, Hurthle cell carcinoma, hyaline carcinoma, hypemephroid carcinoma, infantile embryonal carcinoma, carcinoma in situ, intraepidermal carcinoma, intraepithelial carcinoma, Krompecher's carcinoma, Kulchitzky-cell carcinoma, large-cell carcinoma, lenticular carcinoma, carcinoma lenticulare, lipomatous carcinoma, lymphoepithelial carcinoma, carcinoma medullare, medullary carcinoma, melanotic carcinoma, carcinoma molle, mucinous carcinoma, carcinoma muciparum, carcinoma mucocellulare, mucoepidermoid carcinoma, carcinoma mucosum, mucous carcinoma, carcinoma myxomatodes, nasopharyngeal carcinoma, oat cell carcinoma, carcinoma ossificans, osteoid carcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, schneiderian carcinoma, scirrhous carcinoma, carcinoma scroti, signet-ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tuberous carcinoma, verrucous carcinoma, and carcinoma villosum.

Additional cancers which can be treated with ester according to the invention include, for example, Hodgkin's Disease, Non-Hodgkin's Lymphoma, multiple mycloma, neuroblastoma, breast cancer, ovarian cancer, lung cancer, rhabdomyosarcoma, primary thrombocytosis, primary macroglobulinemia, small-cell lung tumors, primary brain tumors, stomach cancer, colon cancer, malignant pancreatic insulanoma, malignant carcinoid, unnary bladder cancer, premalignant skin lesions, testicular cancer, lymphomas, thyroid cancer, neuroblastoma, esophagcal cancer, genitourinary tract cancer, malignant hypercalcemia, cervical cancer, endometrial cancer, adrenal cortical cancer, and prostate cancer.

The following examples illustrate the preferred methods for the preparation of the compounds of the present invention, which are not intended to limit the scope of the invention thereto.

### EXAMPLES

Chemistry:

Mostly freely water soluble esters of acetylenic amines, easily prepared from cheep and available starting materials have been found to possess anti-tumour activity against several tumour cell lines. They have the general formula:
[N-(4-amino-2-butynyl)carboxylate]
where the amino species are:
diethylamine, diphenylamine, pyrrolidine, piperidine, methylpiprazine, morpholine, thiomorpholine, hexamethylene imine, and the carboxylate species are:
formate, acetate, propanoate, benzoate, cyclohexyl carboxylate, pivalate, have been prepared according to Mannich reaction which includes the reaction of propargyl carboxylate and the secondary amine in dioxane. Some of these esters have been already known and the others are new (prepared for the first time). The known esters have been prepared in this study, first, to complete the series of these esters and second, to study the anti-tumour activity of all of these esters which have not been previously tested. The acetylenic amines have been characterized by their ¹H and ¹³C NMR {δ (ppm) and J (Hz)}, mass spectra, IR spectra {u (cm⁻¹)}, U.V./Vis. spectra. Their specific gravity and refractive index have also been recorded.

### BACKGROUND

A comprehensive investigations about organic compounds containing 2-butyne nucleus, i.e., (X—CH₂—C≡C—CH—X′), with different X & X′ substituents (usually amino groups) have been carried out and their biological activities as muscarinic receptors were examined¹⁻¹⁰. Some research work about the ester of 2-butyne nucleus was reported in the Russian patent (1976)¹¹, in which X represents a terphthalate or metacrylate groups and X′ represents N-morpholino or N-piprydino groups. However, these compounds had been a target for an industrial usage as substrates for the preparation of polymers and copolymers.

On the other hand, our searching in the literature from 1966 up to October 2002 revealed that there were, apparently, few work mentioned about the chemistry and the anti-tumour studies of acetylenic esters related to our compounds (vide infra)¹²⁻¹⁴. We are, therefore, presenting in this report the synthesis, characterization and antitumour activity of several esters of the general formula: [N-(4-amino-2-butynyl)].

Preparation:

The [N-(4-amino-2-butynyl)]esters were prepared by the successive conversion of propargyl alcohol into the corresponding propargyl ester (I) by a simple esterification with a respective organic acid with or without organic solvent. The proprgyl ester was then converted into the corresponding [N-4-amino-2-butynyl)]ester (II) by Mannich condensation with paraformaldehyde, acetic acid or a respective organic acid and Cu₂Cl₂ (as a catalyst) in dioxane as in the following equation:
R=—H,—CH₃,—CH₃CH₂,—C(CH₃)₃,—C₆H₅,—C₆H₁₁
tert.Amine=—N(CH₃)₂,—N(C₆H₅)₂,—N(C₄H₈),—N(C₅H₁₀),—N(C₆H₁₂),—N(C₄H₈O),—N(C₄H₈S),—N(C₄H₈NCH₃)

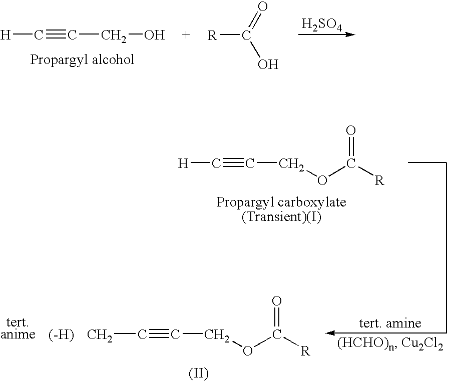

Synthesis of the Transients (I) and the Esters (II)

### EXAMPLES

### Preparation of [N-(4-morphlino-2-butynyl)]acetate (L2)

Step (1). Preparation of Propargyl Acetate:

Propargyl alcohol (28 g, 0.5 mole) was placed into a round bottomed flask fitted with water condenser and glacial acetic acid (30 g, 0.5 mole) was added followed by drop wise addition of concentrated H₂SO₄ (3 ml). The mixture was refluxed under string for ca. 6 h, and by which time the colour turned dark brown. On cooling the reaction mixture to room temperature, the components were poured onto a cold water (150 ml). The mixture was placed into a separating funnel and small portions of sodium bicarbonate (NaHCO₃) as added gradually in order to separate the oil from the aqueous layer. The oily layer was separated from the aqueous one and the latter was treated again with NaHCO₃ until all oils separated. The combined oils was treated with unhydrous MgSO₄ for one day (for drying). The dark brown oil was distilled at 116-118° C. to give colourless oil. Yield=32 g (65%, based on propargyl alcohol used).

Step (2). Conversion of Propargyl Acetate to L2

Propargyl acetate (10 g, 0.1 mole) was mixed with morpholine (10.65 g, 0.12 mole) and the mixture was cooled to 0° C. in a salted ice-bath. A mixture of grinded paraformaldehyde (3.8 g, 0.13 mole) and Cu2Cl2 (0.3 g) in dioxane (80 ml) was added gradually while the temperature was maintained at ca. 0° C. To this was added glacial acetic acid (16 ml) drop wise during a period of ca.10 min. The reaction mixture was became solid-gel, thus a vigorous stirring is necessary to mix all the components. After the addition of acetic acid was completed which the colour turned blue, the cooling was refluxed for ca.3 h. During the heating, the colour of the mixture was changed to deep blue, olive-green, brown-red then dark-red and it was clear in feature in which all the suspended paraformaldehyde was dissolved.

The mixture was cooled to room temperature and cold water (350 ml) was added.

The pH value of the mixture was corrected to 1 by a slow addition of diluted hydrochloric acid (50%) and the acidic mixture was kept in the refrigerator for overnight.

The acidic mixture was extracted twice with ether (150 ml) to remove any impurities. The aqueous layer was treated with small portions of solid NaHCO3 until the pH became 7, then NaOH (1M) was added drop wise until the pH was corrected to 9. The alkaline mixture was extracted twice with chloroform (150 ml) and the organic layer was dried over Na₂CO₃ for overnight. The mixture was filtered to remove Na₂CO₃ and the clear brown organic solution was heated with charcoal then filtered. The filtrate was distilled to evaporate all the chloroform and the remaining red-brown oil was redissolved in ether (100 ml) and filtered. The filtrate was eluted through neutral alumina, 10 cm length and 0.5 cm diameter. Distillation of all ether from the eluant gave yellow-orange oil.

This was distilled under reduced pressure to give colourless to pale yellow oil.

Yield 12 g (60% based on propargyl acetate used).

The esters L7, L11, L13, L19 and L25 were prepared similarly, from the reaction of propargyl acetae with pipyridine, methyl piperazine, thiomorpholine, pyrrolidine and hexamthylene imine, respectively.

### Preparation of [N-(4-morpholino-2-butynyl)]benzoate (L4)

Step (1). Preparation of Propargyl Benzoate:

Propargyl alcohol (14 g, 0.25 mol) was mixed with benzoic acid (30.5 g, 0.25 mol) and concentrated H₂SO₄ (ca.2 ml) in toluene (50 ml). The reaction mixture was stirred under reflux for ca. 2 h. On cooling to room temperature, all the toluene was evaporated and the resulting brown gelly residue was poured onto distilled water (250 ml) and NaHCO₃ was added in small portions to neutralize the solution to pH 7. the organic material was extracted several times with small portions of chloroform and separated by using a separating funnel and the dark brown extract was dried over unhydrous MgSO₄ for overnight. The mixture was filtered and all the CHCl₃ evaporated to leave dark brown oily material. This oil can be purified either by distillation under reduced pressure or dissolving the residue in ether and eluting the solution through silica gel (see below for specification of the gel and the column) and the yellowish ethereal solution thus obtained was evaporated to leave pale yellow oil. This was pumped very hard to give a pure enough product for further processes.

Step (2). Conversion of Propargyl Benzoate to L4

Proparyl benzoate (10 g, 62.5 mmol) was mixed with morpholine (6.5 g, 73 m mol) and placed into 250 ml round bottomed flask with cooling in ice bath. To this was added a mixture of paraformaldehye (2.2 g, 73 mmol) and Cu₂Cl₂ (0.15 g) in dioxane (60 ml). Benzoic acid or acetic acid (73 mmol) in dioxane was gently added to the reaction mixture with vigorous stirring. Cooling was stopped and the reaction mixture was then heated under reflux for ca. 5 h. During which time, the colour was turned blue, dark green, olive green, brown and then dark maroon it was cooled to room temperature and poured onto distilled cold water (ca. 200 ml). The pH value was corrected to 1 by addition of dilute hydrochloric acid (1:1) and the total volume was extracted with ether (3×100 ml). The pH value of the aqueous layer was corrected to 9 by either addition of solid NaHCO₃ until PH=7 followed by addition of NaOH solution (5 M) or directly by careful addition of NaOH solution (5 M) with stirring until the pH value become 9. The alkaline solution was extracted with CHCl₃ (4×150 ml). The organic layer was separated and dried over unhydrous Na₂CO₃ for overnight. Removal of Na₂CO₃ by filtration, and evaporation of all CHCl₃ leave an red-brown residual oil. This Oil was redissolved in ether, treated with activated charcoal, and filtered through filter paper. The filtrate was eluted through alumina column (total length 11 cm) fitted with γ-tonerde Al₂O₃, 20 cm length and 1 cm diameter. On evaporation of all the ether from the eluant, an dense pale yellow oil was obtained. It was pumped very hard to remove any trace of the volatiles to give oily product pure enough for other purposes, but nevertheless, it can be distilled at reduced at pressure.

### Preparation of [N-(-4-morpholino-2-butynyl)]formate (L6)

Step (1). Preparation of Propargyl Formate:

Propargyl alcohol (28 g, 0.5 mol) was mixed with formic acid (26 g, 0.5 mol) followed by slow addition of concentrated H₂SO₄ (3 ml). The mixture was refluxed under stirring for ca.5 h, and by which time the colour turned dark brown.

On cooling the mixture to room temperature, cold water (150 ml) was added and the organic layer was extracted and separated by adding solid NaHCO₃. The organic layer was dried over unhydrous Na2CO₃ for overnight. It was filtered through filter paper and the filtrate was distilled at 102-104° C. to give pale yellow material. Yield=30 g (40% based on propargyl alcohol used).

Step (2). Conversion of Propargyl Formate to L6:

Propargyl formate (10 g, 0.1 mole) was mixed with morpholine (10.15 g, 0.117 mole) and the mixture was cooled to 0° C. in a salted ice-bath. A mixture of grinded paraformaldehyde (3.5 g, 0.117 mole) and Cu2Cl2 (0.25 g) in dioxane (75 ml) was added gradually while the temperature was maintained between 5-0° C. To this was added formic acid or glacial acetic acid (0.25 mole) drop wise during a period of ca. 10 min. The reaction mixture became light blue-green solid-gel; it was stirred vigorously to homogenize the components.

The mixture was then heated under reflux for ca.3 h and during which time the colour changed to green and lighten then to orange and finally to dark brown-green. The mixture was allowed to cool to room temperature and cold water (350 ml) was added. The pH value was corrected to 1 by a slow addition of HCl (50%), then the mixture was extracted twice with ether (150 ml) and the aqueous layer was treated with solid NaHCO₃ until the pH became 7. Further correction of the pH to 7 was curried out by a slow addition of NaOH solution (1 M). The alkaline solution was extracted three times with CHCl₃ (150 ml) and the organic layer was dried over anhydrous Na2CO₃ for overnight. The mixture was filtered and the filtrate was taken to dryness to give brown oil. This was eluted as mentioned before for L2 and the etheral solution was evaporated to leave yellowish oil which is pure enough for further purposes, though can be distilled at reduced pressure.

Yield=10 g (55% based on propargyl formate used)

### Preparation of [N-(4-diethylamino-2-butynyl)]acetate (L9)

Step (1). Preparation of Propargyl Acetate:

This was prepared as mentioned above for L2.

Step (2). Conversion of Propargyl Acetate to L9:

Propargyl acetate (5 g, 0.05 mole) was mixed with diethyl amine (6.5 ml, 0.06 mole), paraformaldehyde (1.9 g, 0.06 mole) and Cu2Cl2 (0.15 g) in dioxane (40 ml). The mixture was cooled to ca. 0° C. and glacial acetic acid (8 ml) was added in small portions with vigorous stirring. The reaction mixture was treated as for the preparation of L2. The remaining yellow-brown oil obtained after all processes was distilled under reduced pressure to give yellow oil.

Yield=6 g (70% based on propargyl acetate used).

### Preparation of [N-(4-diphenylamino-2-butynyl)]acetate (L12)

Step (1). Preparation of Propargyl Acetate:

This was prepared as mentioned above for L2.

Step (2). Conversion of Propargyl Acetate to L12:

Propargyl acetate (2.5 g, 0.03 mole) was mixed with diphenyl amine (5.15 g, 0.03 mole), paraformaldehyde (0.95 g, 0.03 mol) and Cu2Cl2 (0.1 g) in dioxane (40 ml). The mixture was cooled to ca. 0° C. and glacial acetic acid (4 ml) was added in small protions with vigorous stirring. The reaction mixture was treated as for the preparation of L2. The remaining maroon oil obtained after all process was very difficult to distill under reduced pressure, it was then redissolved in ether and treated with charcoal for decolourlization and the filtrate was eluted through alumina to give pale red solution. Evaporation of all ether gave a viscous red-brown oil.

Yield=5 g (62% based on propargyl acetate used).

### Preparation of [N-(4-morpholino-2-butynyl)]propionate (L15) and

### [N-(4-thiomorpholino-2-butyryl)]propionate (L16)

Step (1). Preparation of Propargyl Propionate:

This was prepared in a similar manner to that of propargyl acetate, i.e., from propionic acid and concentrated H2SO4.

Step (2). Conversion of Propargyl Propionate to L15 and L16:

Propargyl propionate was converted into L15 or L16 by a similar method to that for L2 to give colourless to pale yellow oil. Yield=71% for L15 and 65% for L16

### Preparation of [N-(4-morpholino-2-butynyl)]pivalate (L18)

Step (1): Preparation of Propargyl Pivalate:

Propargyl alcohol (12.0 g, 0.2 mole) was mixed with pivalic acid (trimethylacetic acid) (20.4 g, 0.2 mole) and the mixture was cooled to 0° C. temperature. Concentrated H2SO4 (3 ml) was added gradually while the temperature was maintained between 0-5° C. for 30 min. The cooling was then stopped and the mixture was stirred for ca.2 h at room temperature and for ca. 1 h at 70° C. On cooling to room temperature, the mixture turned brown in colour, it was poured onto cold water (150 ml) and treated as for the preparation of propargyl acetate. The product was distilled under reduced pressure to give colourless oil (yield >60%).

Step (2): Conversion of Propargyl Pivalate to L18:

Propargyl pivalate (10 g, 0.07 mole) was mixed with morpholine (7 ml, 0.08 mole), paraformaldelyde (2.5 g, 0.08 mole) and Cu2Cl2 (0.2 g) in dioxane (80 ml). The mixture was cool to ca. 0° C. and pivalic acid (8 ml) or acetic acid (12 ml) was added in small portion with vigorous stirring. The reaction mixture was treated as for the preparation of L12. After decolourizing and elution of the etheral solution through alumina and evaporation of all ether gave a colourless oil (yield=80%).

### Preparation of N,N′-[(4,4′-piprazino)-bis-(2-butynyl)]diacetate (L20)

Step (1). Preparation of Propargyl Acetate:

This was prepared as mentioned previously for L2.

Step (2). Conversion of Propargyl Acetate to L20:

Propargyl acetate (10 g, 0.1 mole) was mixed with anhydrous piprazine (4.6 g, 0.05 mole), paraformaldehyde (3.5 g, 0.1 mole) and Cu2Cl2 (0.3 g) in dioxane (100 ml). The mixture was cooled to ca. 5° C. in which the colour became light blue-green and after gradual addition of glacial acetic acid (16 ml), the mixture became paler in colour and suspension in feature. The mixture was refluxed for ca. 3 h in which the colour turned maroon. On cooling to room temperature, the mixture was poured onto cold water (250 ml) and treated as for the preparation of L2. The final step and after elution of the etheral solution of the oil thus obtained, the clear pale yellow etheral eluant was evaporated gradually. White crystalline material started to deposit and after the precipitation was completed, the crystals were filtered off and washed with small portions of cold ether. The filtrate was combined with the washings and left in the refrigerator for overnight to give another batch of crystals.

Combination of all crops of crystals gave (10 g) of the product (L22), i.e., 64% based on propargyl acetate used.

### Preparation of [N-(4-morpholino-2-butynyl)]cyclohexyl carboxylate (L22)

Step (1): Preparation of Propargyl Cyclohexyl Carboxylate

Propargyl alcohol (14 g, 0.25 mole) was mixed with cyclohexyl carboxylic acid (32 g, 0.25 mole) and concentrated H2SO4 (3 ml). The mixture was refluxed forca.2 h and by which time it became viscous in feature and brown in colour. Cold distilled water (200 ml) was added to the reaction and placed into a separating funnel and NaHCO3 was gradually added until no reaction with acidic solution was observed. The organic layer (oil/solid) was dissolved in CHCl3 (200 ml) and dried over Na₂CO3 for several hours. This was filtered and the red-brown filtrate was boiled with charcoal for several minutes and filtered again. The clear orange solution was taken to dryness to leave an orange oily material. The oil was dissolved in ether and eluted through alumina to give pale yellow solution. Evaporation of all ether gave pale yellow oil. Yield=25 g (60% based on the propargyl alcohol used).

Step (2). Conversion of Propargyl Cyclohexyl Carboxylate to L22:

Propargyl cyclohexyl carboxylate (16.6 g, 0.1 mole) was mixed with morpholine (9.5 g, 0.12 mole), paraformaldehyde (3.6 g, 0.12 mole) and Cu2Cl2 (0.2 g) in dioxane (55 ml). The mixture was cooled to ca. 0° C. and cyclohexyl carboxylic acid or acetic acid (0.12 mole) was added in small portions with vigorous stirring. The reaction mixture was treated as for the preparation of L12.

The last step offered a pale yellow oil. Yield=18.5 g (70% based on propargyl cyclohexyl carboxylate used).

Physical properties of the transient (I)/propargyl carboxylate

1. Propargyl Acetate (R═CH₃): Colourless Oil

| ¹H NMR | ¹³C NMR | λₘₐₓ = 235.5 nm | IR Spectrum | Mass Spectrum |
|---|---|---|---|---|
| δ H = 2.4 t (H) J = 2.5 | δ CH = 51.3 | ²⁰ η = 1.4207 | υ(H—C≡) = 3291.6 s | M/Z = 196 {possibly |
| δ CH₂ = 4.4 d (2H), J = 2.5 | δ CH₂ = 74.6 | | υ(C≡C) = 2130 m | for dimmer compound (its M.W. = |
| δ CH₃ = 1.85 s (3H) | δ CH₃ = 19.8 | | υ(C═O) 1744 s | 98) via hydrogen bonds}. |
| | δ CO = 169.6 | | | Fragments: 154, 125, 107, 92 |

2. Propargyl Benzoate (R═C6H5): Colourless Oil

| ¹H NMR | ¹³C NMR | λₘₐₓ = 238.5 & 274 nm | Mass Spectrum |
|---|---|---|---|
| δ H = 2.6 t (H), J = 2.5 | δ CH = 52.0 | ²⁰ η = 1.5262 | M/Z = 198 |
| δ CH₂ = 4.85 d (2H), J = 2.5 | δ CH₂ = 75.0 | | Fragments: 167, 136, 105 |
| δ C₆H₅ = 7.3 − 7.5 m (3H), | δ C₆H₅: | | |
| 7.9 − 8.1 m (2H) | C1 = 128.9 | | |
| | C2 = 132.9 | | |
| | C3 = 128.0 | | |
| | C4 = 129.3 | | |
| | δ CO = 165.2 | | |

3. Propargyl Formate (R═H): Colourless Oil

| ¹H NMR | ¹³C NMR | λₘₐₓ = 233.5 nm | IR Spectrum: |
|---|---|---|---|
| δ H = 2.6 t (H), J = 3.3 | δ CH = 51.0 | ²⁰ η = 1.415 | υ(H—C≡) = 3295.4 s |
| δ CH₂ = 4.8, (2H) not resolved | δ CH₂ = 75.4 | ²⁰ D = 1.04 | υ(C≡C) = 2130 m |
| doublet | δ CO = 160.2 | | υ(C═O) = 1744, 1647.7 s |
| δ HCO = 8.07 t (H), J = 3 | | | |

4. Propargyl Propionate (R═CH₃CH₂): Colourless Oil

| ¹H NMR | λₘₐₓ = 230.5 nm |
|---|---|
| δ H = 2.5 t (H), J = 2.0 | ²⁰ η = 1.4220 |
| δ CH₂O = 4.7 d (2H), J = 2.0 | ²⁰ D = 0.93 |
| δ CH₃ = 1.16 t (3H), J = 7.6 | |
| δ CH₂ = 2.4 q, (2H), J = 7.2 | |

5. Propargyl Trimethyl Acetate (Pivalate) {R═C(CH₃)₃}: Colourless Oil

| ¹H NMR | λₘₐₓ = 231.0 nm |
|---|---|
| δ H = 2.45 t (H), J = 1.8 | ²⁰ η = 1.4180 |
| δ CH₂ = 4.66 d (2H), J = 2.7 | |
| δ CH₃ = 1.2 s (9H) | |

6. Propargyl Cyclohexyl Carboxylate (R═C₆H₁₁): Pale Yellow Oil

| ¹H NMR | λₘₐₓ = 233.5 nm |
|---|---|
| δ H = 2.46 t (H), J = 2.0 | ²⁰ η = 1.4650 |
| δ CH₂ = 4.65 d (2H), J = 1.6 | |
| δ C₆H₁₁ = 1.3 − 2.35 m (11H) | |

Physical Properties of the Esters (II)/L Series

L2

Structure: 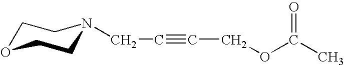
Name: N-(4-morpholino-2-butynyl)acetate
Feature and Colour: Colourless to pale yellow oil

λₘₐₓ=236.5 nm, η²⁰=1.4855, D²⁰=1.087 g ml⁻¹

| ¹H NMR | ¹³C NMR | Mass Spectrum: | IR Spectrum: |
|---|---|---|---|
| δ CH₃ = 1.60 s (3H) | δ CH₃ = 19.3 | M/Z = 197 | υ(C≡C) = 2000 w |
| δ (CH₂—N) = 2.84 t (2H), J = 2.0 | δ (CH₂—N) = 46 | Fragments: 154, 137, | υ(C═O) = 1744, 1647.7 s |
| δ (CH₂—O) = 4.23 t (2H), J = 2.0 | δ (CH₂—O) = 50.9 | 124, 108, 86, 56 | |
| δ N(CH₂)₂ = 2.03 q (4H) | δ N(CH₂)₂ = 51.1 | | |
| δ O(CH₂)₂ = 3.20 q (4H) | δ O(CH₂)₂ = 65.5 | | |
| | δ (N . . . C≡) = 78.4 | | |
| | δ (O . . . C≡) = 80.5 | | |
| | δ CO = 168.6 | | |

Mass Spectrum:

M/Z=197

Fragments: 154, 137, 124, 108, 86, 56

IR Spectrum:

[symbol] (C≡C)=2000 w

[symbol] (C═O)=1744, 1647.7 s

L4

Structure: 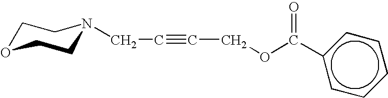
Name: N-(4-morpholino-2-butynyl)benzoate
Feature and Colour: Yellow oil

λmax=240 and 273.5 nm, η²⁵=1.5411, D²⁵=1.066 g ml⁻¹

| ¹H NMR | ¹³C NMR | Mass Spectrum: | IR Spectrum: |
|---|---|---|---|
| δ C₆H₅ = 7.2 − 7.9 m (5H) | δ C₆H₅: C1 = 129, C2 = 132.65, | M/Z = 259 | υ(C≡C) = 1972 w |
| δ (CH₂—N) = 3.1 t (2H), J = 2.0 | C3 = 127.85, C4 = 129.2 | Fragments: 137, 99 | υ(C═O) = 1725 s |
| δ (CH₂—O) = 4.8 t (2H), J = 2.0 | δ (CH₂—N) = 46.8 | | |
| δ N(CH₂)₂ = 2.3 q (4H) | δ (CH₂—O) = 52.2 | | |
| δ O(CH₂)₂ = 3.5 q (4H) | δ N(CH₂)₂ = 51.7 | | |
| | δ O(CH₂)₂ = 66.1 | | |
| | δ (N . . . C≡) = 78.9 | | |
| | δ (0 . . . C≡) = 81.3 | | |
| | δ CO = 165.0 | | |

Mass Spectrum:

M/Z=259

Fragments: 137, 99

IR Spectrum:

[symbol] (C≡C)=1972 w

[symbol] (C═O)=1725 s

L6

Structure: 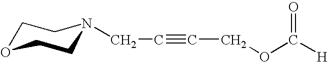
Name: N-(4-morpholino-2-butynyl)formate
Feature and Colour: Pale yellow oil

λₘₐₓ=236 nm, η^{20=1.4956}, D²⁰=1.13 g ml⁻¹

| ¹H NMR | ¹³C NMR | IR Spectrum: |
|---|---|---|
| δ HC═O = 7.9 b (1H) | δ (CH₂—N) = 40.6 | υ(C≡C) = 1964 w |
| δ (CH₂—N) = 3.26 b (2H) | δ (CH₂—O) = 45.8 | υ(C═O) = 1663 s, |
| δ (CH₂—O) = 4.1 b (2H) | δ N(CH₂)₂ = 52.2 | b |
| δ N(CH₂)₂ = 2.42 b (4H) | δ O(CH₂)₂ = 66.6 | |
| δ O(CH₂)₂ = 3.56 (4H) | δ (N . . . C≡) = 79.1 | |
| | δ (O . . . C≡) = 84.4 | |
| | δ C = 161.0 | |

IR Spectrum:

[symbol] (C≡C)=1964 w

[symbol] (C═O)=1663 s, b

L7

Structure: 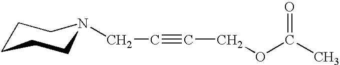
Name: N-(4-piperidino-2-butynyl)acetate
Feature and Colour: Pale yellow oil

λₘₐₓ=232 nm, η²⁰=1.4850, D²⁰=not recorded

| ¹H NMR | ¹³C NMR | IR Spectrum: |
|---|---|---|
| δ CH₃ = 2.1 s (3H) | δ CH₃ = 19.9 | υ(C≡C) = 1980 w |
| δ (CH₂—N) = 3.3 t (2H), J = 1.7 | δ (CH₂—N) = 47.4 | υ(C═O) = 1744, 1647.7 s |
| δ (CH₂—O) = 4.7 t (2H), J = 1.6 | δ (CH₂—O) = 51.4 | |
| δ N(CH₂)_{1, 2} = 2.5 q (4H) | δ N(CH₂)₂ = 53.0 | |
| δ (CH₂)₃₋₅ = 1.6 m (6H) | δ (CH₂)₃ = 27.1 | |
| | δ (N . . . C≡) = 78.6 | |
| | δ (O . . . C≡) = 80.8 | |
| | δ CO = 169.0 | |

IR Spectrum:

[symbol] (C≡C)=1980 w

[symbol] (C═O)=1744, 1647.7 s

L9

Structure: 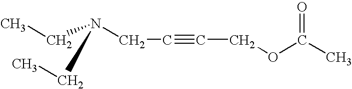
Name: N-[4-(N,N-diethylamino)-2-butynyl]acetate
Feature and Colour: Yellow-orange oil

λₘₐₓ=231.5 nm, η²⁰=1.4732, D²⁰=0.92 g ml⁻¹

| ¹H NMR | ¹³C NMR | IR Spectrum: |
|---|---|---|
| δ CH₃ = 2.1 s (3H) | δ CH₃ = 20.6 | υ(C≡C) = 2000 w |
| δ (CH₂ − N) = 3.4 t (2H), | δ (CH₂ − N) = 41.2 | υ(C = O) = 1752 s |
| J = 1.7 | δ (CH₂ − O) = 52.4 | |
| δ (CH₂ − O) = 4.27 t (2H), | δ (N . . . C≡) = 78.8 | |
| J = 1.8 | δ (O . . . C≡) = 82.1 | |
| δ C₂H₅: | δ CO = 169.9 | |
| CH₃ = 1.1 t (3H), J = 7.5 | δ CH₃CH₂ = 12.6 | |
| CH₂ = 2.45 q (2H), J = 7.0 | δ CH₃CH₂ = 47.3 | |

IR Spectrum:

[symbol] (C≡C)=2000 w

[symbol] (C═O)=1752 s

L11

Structure: 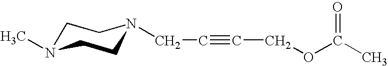
Name: N-[4-(N-methylpiprazino)-2-butynyl]acetate
Feature and Colour: Pale yellow oil

λₘₐₓ=231.5 nm, η²⁰=1.4890, D²⁰=1.01 g ml⁻¹

| ¹H NMR | ¹³C NMR | IR Spectrum: |
|---|---|---|
| δ CH₃C = O = 2.1 s (3H) | δ CH₃C = O = 19.7 | υ(C≡C) = 1985 w |
| δ (CH₂ − N) = 2.3 t (2H), J = 1.7 | δ (CH₂ − N) = 46.1 | υ(C = O) = 1744, 1651.6 s |
| δ (CH₂ − O) = 3.3 t (2H), J = 1.8 | δ (CH₂ − O) = 51.3 | |
| δ N(CH₂)₄ = 2.5 m (8H) | δ N(CH₂)₂ . . . CH₂ = 50.9 | |
| δ O(CH₂)₂ = 3.20 q (4H) | δ N(CH₂)₂ . . . CH₃ = 54.0 | |
| δ CH₃N = 2.3 s (3H) | δ (N . . . C≡) = 78.3 | |
| | δ (O . . . C≡) = 80.9 | |
| | δ CH₃N = 45.1 | |
| | δ CO = 168.6 | |

IR Spectrum:

[symbol] (C≡C)=1985 w

[symbol] (C═O)=1744, 1651.6 s

L12

Structure: 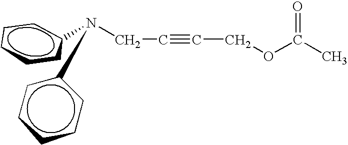
Name: N-[4-(N,N-diphenylamino)-2-butynyl]acetate
Feature and Colour: Red-brown viscous oil

λₘₐₓ=230.5 and 268.5 nm, η²⁰=1.6073, D²⁰=1.0 g ml⁻¹

| ¹H NMR | ¹³C NMR | IR Spectrum: |
|---|---|---|
| δ CH₃ = 2.05 s (3H) | δ CH₃ = 20.6 | υ(C≡C) = 1944 w |
| δ (CH₂ − N) = 4.4 t (2H), J = 1.8 | δ (CH₂ − N) = 42.2 | υ(C = O) = 1744 s |
| δ (CH₂ − O) = 4.65 t (2H), J = 1.7 | δ (CH₂ − O) = 52.3 | |
| δ C₆H₅ = 7.0 − 7.4 m (10H) | δ (N . . . C≡) = 78.0 | |
| | δ (O . . . C≡) = 82.9 | |
| | δ CO = 170.2 | |
| | δ C₆H₅: C1 = 147.4, C2 = 129.3, | |
| | C3 = 121.2, C4 = 122.2 | |

IR Spectrum:

[symbol] (C≡C)=1944 w

[symbol] (C═O)=1744 s

L13

Structure 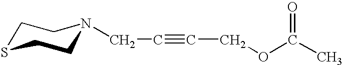
Name: N-(4-thiomorpholino-2-butynyl)acetate
Feature and Colour: Pale yellow oil

λₘₐₓ=231.5 nm, η²⁵=1.5275, D²⁵ 1.13 g ml⁻¹

| ¹H NMR | | | ¹³C NMR | Mass Spectrum: | IR Spectrum: |
|---|---|---|---|---|---|
| δ CH₃ = 2.1 s (3H) | | | δ CH₃ = 20.6 | M/Z = 213 | υ(C≡C) = 1978 w |
| δ (CH₂ − N) = 3.3 t (2H), J = 1.8 | | | δ (CH₂ − N) = 48.4 | Fragments: 171, 143, 108 | υ(C = O) = 1744 s |
| δ (CH₂ − O) = 4.7 t (2H), J = 1.8 | | | δ (CH₂ − O) = 52.3 | | |
| δ N(CH₂)₂ | | | δ N(CH₂)₂ = 54.0 | | |
| | {close oversize brace} | = 2.75 b, i (8H) | δ S(CH₂)₂ = 67.1 | | |
| δ S(CH₂)₂ | | | δ (N . . . C≡) = 79.5 | | |
| | | | δ (O . . . C≡) = 81.8 | | |
| | | | δ CO = 169.9 | | |

Mass Spectrum:

M/Z=213

Fragments: 171, 143, 108

IR Spectrum:

[symbol] (C≡C)=1978 w

[symbol] (C═O)=1744 s

L15

Structure: 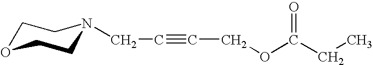
Name: N-(4-morpholino-2-butynyl)propionate
Feature and Colour: Pale yellow oil

λₘₐₓ=231.5 nm, η²⁰=1.4810, D²⁰=1.05 g ml⁻¹

| ¹H NMR | ¹³C NMR | Mass Spectrum: | IR Spectrum: |
|---|---|---|---|
| δ CH₃ = 1.16 t (3H), J = 7.6 | δ CH₃CH₂ = 9.0 | M/Z = 211 | υ(C≡C) = 1975.7 w |
| δ CH₂ = 2.36 q (2H), J = 7.6 | δ CH₃CH₂ = 27.4 | Fragments: 155, 137, 108, 86, 57 | υ(C = O) = 1740.4 s |
| δ (CH₂ − N) = 3.3 t (2H), J = 1.8 | δ (CH₂ − N) = 47.5 | | |
| δ (CH₂ − O) = 4.23 t (2H), J = 1.8 | δ (CH₂ − O) = 52.1 | | |
| δ N(CH₂ = 2.55 q (4H), J = 1.8, 2.7 | δ N(CH₂)₂ = 52.4 | | |
| δ O(CH₂)₂ = 3.7 q (4H), J = 1.8, 2.7 | δ O(CH₂)₂ = 66.8 | | |
| | δ (N . . . C≡) = 79.6 | | |
| | δ (O . . . C≡) = 81.6 | | |
| | δ CO = 173.35 | | |

Mass Spectrum:

M/Z=211

Fragments: 155, 137, 108, 86, 57

IR Spectrum:

[symbol] (C≡C)=1975.7 w

[symbol] (C═O)=1740.4 s

L16

Structure: 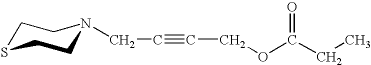
Name: N-(4 thiomorpholino-2-butynyl)propionate
Feature and Colour: Colourless oil

λₘₐₓ=233.5 nm, η²⁵=1.5150, D²⁰=not recorded

| ¹H NMR | | | ¹³C NMR | IR Spectrum: |
|---|---|---|---|---|
| δ CH₃ = 1.16 t (3H), J = 7.5 | | | δ CH₃CH₂ = 9.0 | υ(C≡C) = 1985 w |
| δ CH₂ = 2.36 q (2H),J = 7.6 | | | δ CH₃CH₂ = 27.4 | υ(C = O) = 1745 s |
| δ (CH₂ − N) = 3.3 t (2H), J = 1.9 | | | δ (CH₂ − N) = 48.4 | |
| δ (CH₂ − O) = 4.7 t (2H), J = 1.8 | | | δ (CH₂ − O) = 52.2 | |
| 67 N(CH₂)₂ | | | δ N(CH₂)₂ = 54.0 | |
| | {close oversize brace} | = 2.75 b, i (8H) | δ O(CH₂ = 67.2 | |
| δ S(CH₂)₂ | | | δ (N . . . C≡) = 79.7 | |
| | | | δ (O . . . C≡) = 81.7 | |
| | | | δ CO = 173.4 | |

IR Spectrum:

[symbol] (C≡C)=1985 w

[symbol] (C═O)=1745 s

L18

Structure: 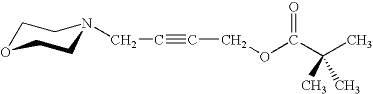
Name: N-(4-morpholino-2-butynyl)pivalate
Feature and Colour: Colourless oil

λₘₐₓ=237.0 nm, η²⁵=1.4710, D²⁵=1.0 g ml⁻¹

| ¹H NMR | ¹³C NMR | Mass Spectrum: | IR Spectrum: |
|---|---|---|---|
| δ CH₃ = 1.20 s (9H) | δ (CH₂)₃C = 27.0, δ (CH₃)₃ C = 81.3 | M/Z = 239 | υ(C≡C) = 1980 w |
| δ (CH₂ − N) = 3.31 t (2H), J = 2.0 | δ (CH₂ − N) = 47.5 | Fragments: 154, 137, 108, 86, 57 | υ(C = O) = 1736.5 s |
| δ (CH₂ − O) = 4.68 t (2H), J = 2.0 | δ (CH₂ − O) = 52.1 | | |
| δ N(CH₂)₂ = 2.53 q (4H) | δ N(CH₂)₂ = 52.4 | | |
| δ O(CH₂)₂ = 3.73 q (4H) | δ O(CH₂)₂ = 66.8 | | |
| | δ (N . . . C≡) = 79.8 | | |
| | δ (O . . . C≡) = 81.8 | | |
| | δ CO = 177.4 | | |

Mass Spectrum:

M/Z=239

Fragments: 154, 137, 108, 86, 57

IR Spectrum:

[symbol] (C≡C)=1980 w

[symbol] (C═O)=1736.5 s

L19

Structure: 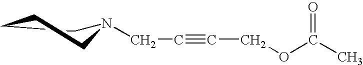
Name: N-(4-pyrrolidino-2-butynyl)acetate
Feature and Colour: Pale yellow oil

λₘₐₓ=231.0 nm, η²⁰=1.4750, D²⁰ 1.13 g ml⁻¹

| ¹H NMR | ¹³C NMR | IR Spectrum: |
|---|---|---|
| δ CH₃ = 2.1 s (3H) | δ CH₃ = 20.6 | υ(C≡C) = 2000 w |
| δ (CH₂ − N) = 3.7 t (2H), | δ (CH₂ − N) = 43.3 | υ(C = O) = 1744, |
| J = 1.7 | δ (CH₂ − O) = 52.3 | 1644 s |
| δ (CH₂ − O) = 4.7 t (2H), | δ N(CH₂)₂ = 52.35 | |
| J = 1.7 | δ (CH₂)₂ = 24.0 | |
| δ N(CH₂ = 2.6 q (4H) | δ (N . . . C≡) = 67.2 | |
| δ (CH₂)₂ = 1.8 q (4H) | δ (O . . . C≡) = 82.9 | |
| | δ CO = 170.2 | |

IR Spectrum:

[symbol] (C≡C)=2000 w

[symbol] (C═O)=1744, 1644 s

L20

Structure: 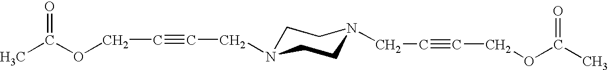
Name: N,N′-[(4,4′-piprazino)-bis-(2-butynyl)]diacetate
Feature and Colour: White solid

λₘₐₓ=231.5 nm, m.p.=69-72° C.

| ¹H NMR | ¹³C NMR |
|---|---|
| δ CH₃ = 2.1 s (6H) | δ CH₃ = 20.6 |
| δ (CH₂ − N) = 3.33 t (4H), J = 1.7 | δ (CH₂ − N) = 47.15 |
| δ (CH₂ − O) = 4.7 t (4H), J = 1.6 | δ (CH₂ − O) = 52.4 |
| δ 2N(CH₂)₂ = 2.63 q (8H), J = 1.6 | δ 2N(CH₂)₂ = 51.95 |
| | δ (N . . . C≡) = 78.8 |
| | δ (O . . . C≡) = 80.0 |
| | δ CO = 170.1 |

CHN Analysis:

| | % C | % H | % N | IR Spectrum: |
|---|---|---|---|---|
| Found: | 62.62 | 7.01 | 9.12 | υ(C≡C) = 1972 w |
| Calc. for C₁₆H₂₂O₄N_{2:} | 62.75 | 7.19 | 9.15 | υ(C = O) = 1740 s |

IR Spectrum:

[symbol] (C≡C)=1972 w

[symbol] (C═O)=1740 s

L22

Structure: 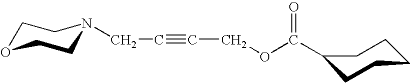
Name: N-(4-morpholino-2-butynyl)cyclohexyl carboxylate
Feature and Colour: Pale yellow oil

λₘₐₓ=231.5 nm, η²⁰=1.4870, D²⁰ 1.03 g ml⁻¹

| ¹H NMR | ¹³C NMR | IR Spectrum: |
|---|---|---|
| δ C₆H₁₁ = 1.3 − 2.1 m (11H) | δ C₆H₁₁: C1 = 67.1, C2 = 43.0, | υ(C≡C) = 1975.7 w |
| δ (CH₂ − N) = 3.32 t (2R), J = 1.4 | C3 = 25.9, C4 = 25.5 | υ(C = O) = 1740 s |
| δ (CH₂ − O) = 4.69 t (2H), J = 1.4 | δ (CH_{2,} − N) = 47.5 | |
| δ N(CH₂)₂ = 2.54 q (4H), J = | δ (CH_{2,} − O) = 52.0 | |
| δ O(CH₂)₂ = 3.70 q (4H), J = | δ N(CH₂)₂ = 52.4 | |
| | δ O(CH₂)₂ = 66.8 | |
| | δ (N . . . C≡) = 79.8 | |
| | δ (O. . . C≡) = 81.5 | |
| | δ CO = 174.8 | |

IR Spectrum:

[symbol] (C≡C)=1975.7 w

[symbol] (C═O)=1740 s

L25

Structure: 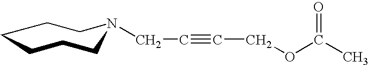
Name: N-[4-(hexamethyleneimino)-2-utynyl)acetate.2dioxane*
The compound is solvated with two moles of dioxane.
   Feature and Colour: Colourless to pale yellow oil

λₘₐₓ=231.5 nm, η²⁰=1.4835, D²⁵=0.982 g ml⁻¹

| ¹H NMR | ¹³C NMR | IR Spectrum: |
|---|---|---|
| δ CH₃ = 2.1 s (3H) | δ CH₃ = 20.7 | υ(C≡C) =2053 w |
| δ (CH₂ − N) = 3.4 t (2H), J = 6.4 | δ (CH₂ − N) = 48.4 | υ(C = O) = 1748, 1647.7 |
| δ (CH₂ − O) = 4.7 t (2H), J = 6.5 | δ (CH₂ − O) = 52.5 | |
| δ N(CH₂)₂ = 2.67 m (4H) | δ N(CH₂)₂ = 55.2 | |
| δ (CH₂)₄ = 1.6 b (8H) | δ 2(CH₂)₂ = 26.9, 28.4 | |
| δ (dioxane) = 3.7 s (16H) | δ (N . . . C≡) = 5.8 | |
| | δ (O . . . C≡) = 83.5 | |
| | δ CO = 170.0 | |
| | δ (dioxane) = 6 7.2 | |

IR Spectrum:

[symbol] (C≡C)=2053 w

[symbol] (C═O)=1748, 1647.7

Anti-Cancer Activity of 4-(N-substituted amino)-2-butynyl-1-ester of the Invention

Results of Anti-Cancer Activity

The esters of the invention showed a good anti-cancer activity in the panel of human tumour cell lines. For the following test the following cell lines have been used:

BxPC3 (pancreatic cancer)

Colo 205 (colonic cancer)

ES2 (ovarian cancer)

T47D (breast cancer)

ASTRO373 (glioma)

CRO2 (carcinoid)

Panc-1 (pancreatic cancer)

ACHN (renal cell cancer)

Colo201 (colonic cancer)

Materials and Methods

Cell lines. Except indicated otherwise all cell lines were obtained from the American Type Culture Collection (ATCC, Rockville, Md.) and grown to confluent monolayers in RPMI-1640 bicarbonate medium (Seromed, Berlin, Germany) in a humidified incubator (5% CO₂, 37° C.). Cells were checked for mycoplasma contamination. The medium was supplemented with 10% heat-inactivated fetal bovine serum (Seromed) and 4 mM glutamine. The cells were subcultured by trypsination (0.03% trypsin containing 0.02% EDTA, three times a week). Cell numbers were counted using a TOA Sysmex microcellcounter (TOA, Tokyo, Japan).

Chemicals and solutions. Unless otherwise mentioned, all chemicals were obtained from Sigma (St. Louis, Mo.). Compounds to be tested were used as supplied. CHP was used as 5 mg/ml stock solution in PBS (phosphate buffered saline, Dulbecco) and aliquots were stored frozen at −20° C. Related compounds were used as 2 mg/ml stock solution and stored frozen at −20° C.

Procedure

Cells were distributed to the wells of 96 well microtiter plates (10000 cells/well) in tissue culture medium (RPMI-1640 supplemented with 10% fetal bovine serum and 4 mM glutamine) and medium added for controls. Test compounds were added in the reported dilution steps and the reported dilution steps and the plates incubated for 4 days under tissue culture conditions (37° C., 5% CO₂).

Following incubation proliferation was measured using a tetrazolium-based assay (EZ4U kit, Biomedica, Vienna, Austria). Optical densities (OD) of the individual wells was determined with the help of an ELISA-Reader (all values done in triplicate) and the values obtained in medium controls set to 100% (OD₄₉₀ₙₘ=0.5-1.5).

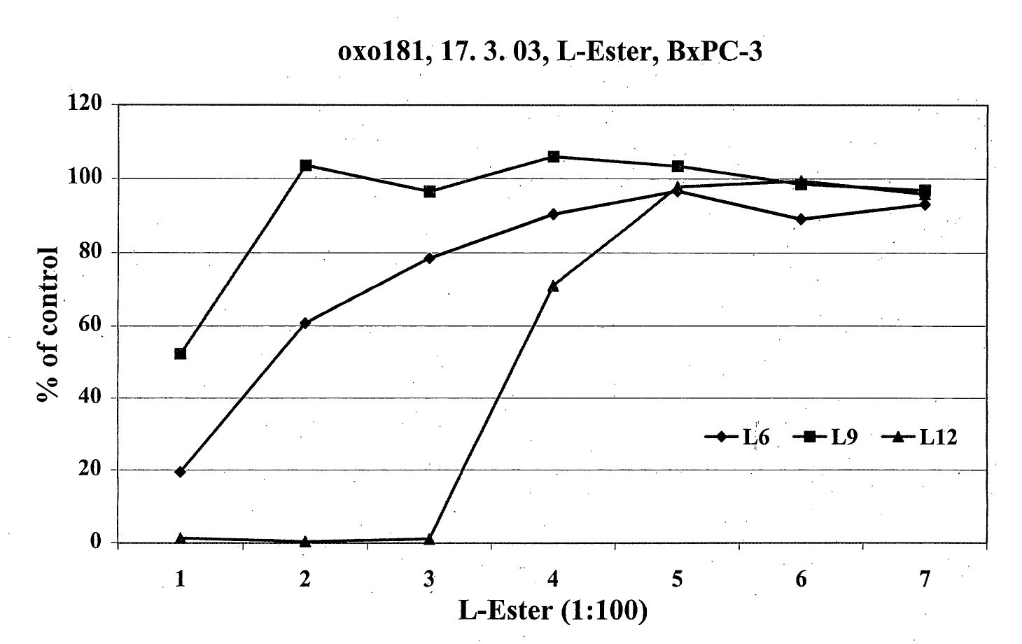

Values given in the figures are % inhibition of the cellular proliferation for the given dilution/con-centrations. The results of the anti-cancer activities of the esters of invention are shown in FIG. 1-4.

FIG. 1=oxo181: examples of dilution curves for L6, L9, L12

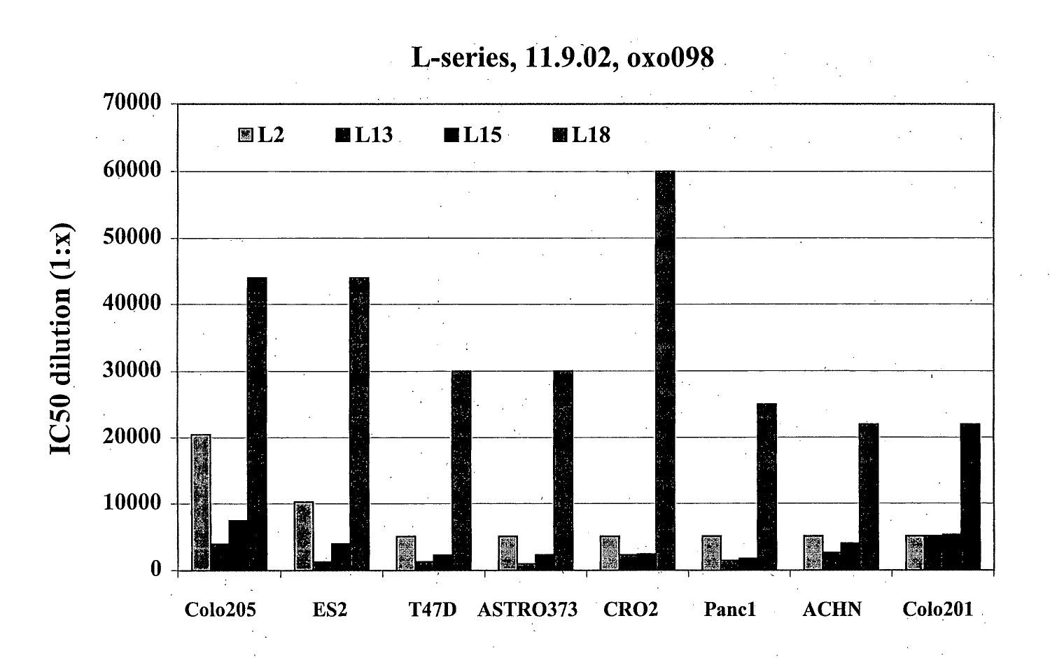

FIG. 2=oxo098: examples L2, L13, L15, L18 of the ester of invention cell line screening

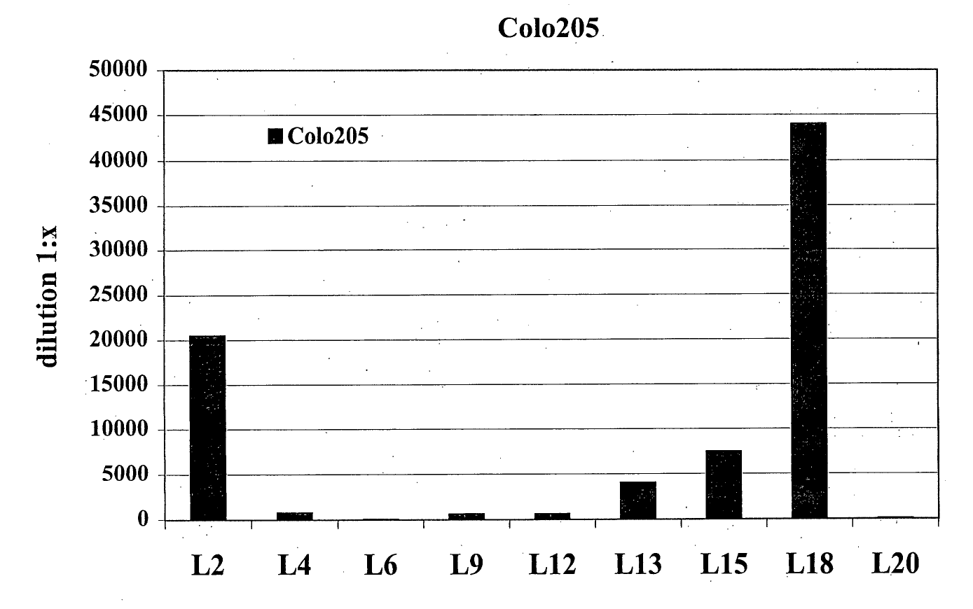

FIG. 3=L-esters: results of all L-esters tested on colo205 cells (colonic cancer)

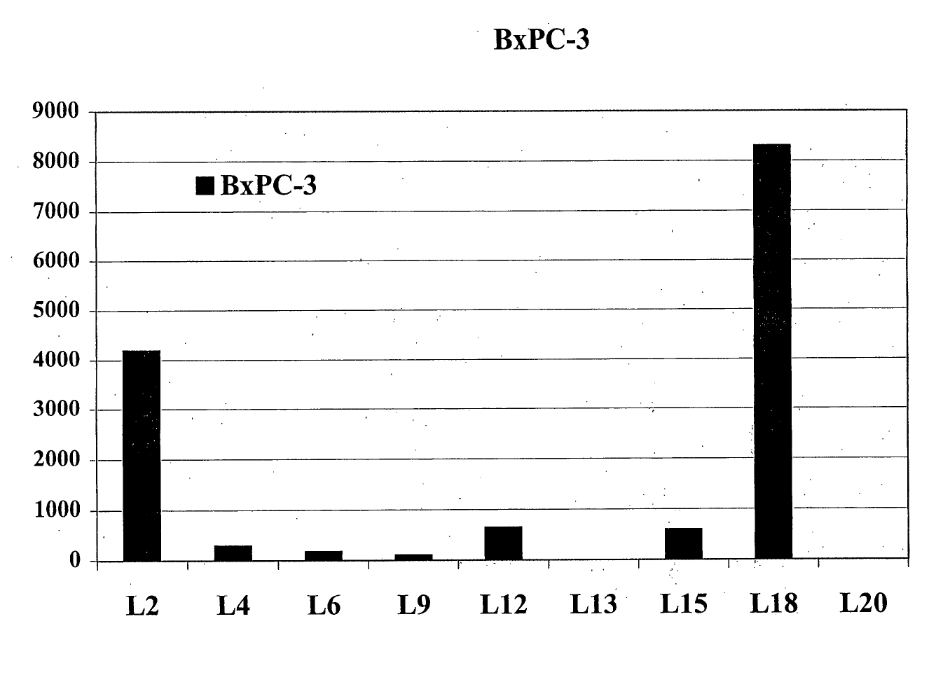

FIG. 4=L-esters: results of all L-esters tested on BxPC3 (pancreatic cancer).

In addition the toxicity of a number of the esters of the invention have been tested on normal cells, Fibroblast (Flow 4000, lung fibroblasts).

Results
% Inhibition of growth at a concentration of 1:400

| | | | |
|---|---|---|---|
| L2 | 18% | L12 | −7.6% |
| L4 | 34.2% | L13 | 19% |
| L6 | 37.4% | L15 | 13% |
| L9 | 5.4% | L18 | 7% |

The esters of the invention L2 [N-(4-morpholino-2-butynyl)acetate] and L18 [N-(4-morpholino-2-butynyl)pivalate]show the highest antitumor activity and low toxicity to fibroblasts.

While the invention has been described in terms of preferred embodiments, the skilled artisan will appreciate that various modifications, substitutions, omissions and changes may be made without departing from the spirit thereof. Accordingly, it is intended that the scope of the present invention be limited solely by the scope of the following claims, including equivalents thereof. Therefore, as will be apparent to those skilled in the art in which the invention is addressed, the present invention may be embodied in forms other than those specifically disclosed above without departing from the spirit or essential characteristics of the invention. The particular embodiments of the present invention, described above, are therefore to be considered in all respects as illustrative and not restrictive. The scope of the present invention is as set forth in the appended claims rather than being limited to the examples contained in the foregoing description.

### LITERATURE

1. B. J. Bradbury, J. Baumgold, R. Paek, U. Kammula, J. Zimmet and K. A. Jacobson, J. Med. Chem., 34(3), 1073 (1991).
2. E. J. Trybulski, R. H. Kramss, R. M. Mangano and A Rusinko, J. Med. Chem., 33(12), 3190 (1990).
3. V. H. Sethy and D. K. Hyslop, Neuropharmacology, 29(2), 185 (1990).
4. J. J. Waite and D. J. Jenden, J. Pharmcol. Exp. Ther., 248(1), 111 (1989).
5. B. Ringdahl, Eur. J. Pharmacol., 140(1), 13 (1987).
6. R. Amstutz, B. Ringdahl, B. Karlen, M. Roch and D. J. Jenden, J. Med. Chem., 28(12), 1760 (1985).
7. N. Hymowitz and H. E. Brezenott, Pharmacol. Biochem. Behav., 21(1), 109 (1984).
8. B. Resul, B. Ringdahl, U. Hacksell, U. Svensson and R. Dahlbom, Acta Pharm. Suec., 16 (4), 225 (1979).
9. A. Lindquist and R. Dahlbom, Dent. Assist., 39 (10), 527 (1970).
10. R. Dahlbom, B. Karlenand A. Lindquist, Acta Pharm. Suec., 3(3), 187 (1966).
11. A. G. Mahsumov, A. Safolev, A. F. Trifonova, S.

H. U. Abdullaev, M. A. Korshunov and F. N. Bondaruk, USSR Patent; SU 248687, Dec. 11, 1976.
12. a) J. M. A. AL-Rawi, A. H. Kuthier and F. T. bachi, Spectrochimica Acta, 43A, 1121 (1987) b) J. L. Neumeyer, U. V. Moyer, J. A. Richman, F. J. Rosenbery and D. G. Teiger, J. Med. Chem., 10(4), 614 (1967). c) D. R. Newell, R. J. Maxwell and B. T. Golding, NMR Biomed., 5, 273 (1992).
13. B. Danielsson, C. Bogentoft and F. Ljungner, Acta Pherm. Suecica, 5, 279 (1998).
14. P. Bador, J. Chantepie, J. Parisand G. Quash, Arzneim.-Forsch./Drug Res. 40(II) (10), 1135 (1990).

1. 4-(N-substituted amino)-2-butynyl-1-esters represented by the following general formula I, their bis-(2-butynyl)diesters and pharmaceutically acceptable salts thereof, 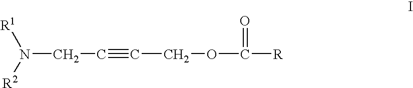
wherein
R is a hydrogen atom; a straight-chained or branched, saturated or unsaturated aliphatic radical with 1-20 C-atoms which is unsubstituted or substituted one or more times by C₁-C₆-alkyl, C₁-C₆-alkoxy, halogen, epoxy, amino, mercapto, a phenyl ring which is unsubstituted or substituted one or more times by C₁-C₆-alkyl, C₁-C₆-alkoxy, hydroxy, epoxy, amino, mercapto or halogen; a cycloalkyl group with 4 to 7 atoms unsubstituted or substituted one or more times by C₁-C₆-alkyl, C₁-C₆-alkoxy, hydroxy, epoxy, amino, mercapto or halogen,
R¹ and R² are joined to form a heterocyclic ring with 3 to 6 C-atoms, unsubstituted or substituted one or more times by C₁-C₆-alkyl, C₁-C₆-alkoxy, hydroxy, halogen, epoxy, amino, mercapto, whereby at least one C-atom can be replaced by O, S or N, or
R¹ and R² are the same or different a hydrogen atom, a straight-chained or branched, saturated or unsaturated aliphatic radical with 1-20 C-atoms, unsubstituted or substituted one or more times by C₁-C₆-alkyl, C₁-C₆-alkoxy, hydroxy, halogen, epoxy, amino or mercapto.

2. 4-(N-substituted amino)-2-butynyl-1-esters according to claim 1,
wherein
R is a hydrogen atom, a straight-chained or branched alkyl group with 1-12 C-atoms, which can be substituted one or more times by C₁-C₆-alkyl; a phenyl ring which can be substituted one or more times by C₁-C₆-alkyl; a cyclo alkyl ring with 5-6 C-atoms which can be substituted one or more times by C₁-C₆-alkyl.

3. 4-(N-substituted amino)-2-butynyl-1-esters according to claim 1,
wherein
R¹ and R² are the same alkyl group with 1-12 C-atoms, which can be straight-chained or branched and substituted by C₁-C₆-alkyl, or
R¹ and R² are joined to form a heterocyclic ring with 4 to 6 C-atoms, whereby at least one C-atom can be replaced by 0, S or N, and the ring can be substituted by C₁-C₆-alkyl.

4. 4-(N-substituted amino)-2-butynyl-1-esters according to claim 1,
wherein
R is a hydrogen atom, a straight-chained or branched alkyl group with 1-6 C-atoms, which can be substituted one or more times by C₁-C₆-alkyl; a phenyl ring which can be substituted one or more times by C₁-C₆-alkyl; a cyclo alkyl ring with 5-6 C-atoms which can be substituted one or more times by C₁-C₆-alkyl, and
R¹ and R² are the same alkyl group with 1-6 C-atoms, which can be straight-chained or branched and substituted by C₁-C₆-alkyl, or
R¹ and R² are joined to form a heterocyclic ring with 4 to 6 C-atoms, whereby at least one C-atom can be replaced by 0, S or N, and the ring can be substituted by C₁-C₆-alkyl.

5. 4-(N-substituted amino)-2-butynyl-1-esters according to claims 4,
wherein
R is H or alkyl and
R¹ and R² are identically methyl, ethyl, propyl, butyl or phenyl; or form together with the N-atom a piperidino, pyrrolidino, morpholino, thiomorpholino, hexamethylene imino, piperazino and methyl piperazino ring.

6. 4-(N-substituted amino)-2-butynyl-1-esters according to claim 5,
wherein 4-(N-substituted amino)-2-butynyl-1-esters are selected from the group comprising
[N-(4-morpholino-2-butynyl)]acetate
[N-(4-piperidino-2-butynyl)]acetate
[N-(4-(N-methyl piperazino-2-butynyl)]acetate
[N-(4-thiomorpholino-2-butynyl)]acetate
[N-(4-pyrrolidino-2-butynyl)]acetate
[N-(4-hexamethylene imino-2-butynyl)]acetate
[N-(4-morpholino-2-butynyl)]benzoate
[N-(4-morpholino-2-butynyl)]formate
[N-(4-diethylamino-2-butynyl)]acetate
[N-(4-diphenylamino-2-butynyl)]acetate
[N-(4-morpholino-2-butynyl)]propionate
[N-(4-thiomorpholino-2-butynyl)]propionate
[N-(4-morpholino-2-butynyl)]pivalate
[N,N′-(4,4-piperazino-bis-2-butynyl)]diacetate and
[N-(4-morpholino-2-butynyl)]cyclohexyl carboxylate.

7. Method for producing 4-(N-substituted amino)-2-butynyl-1-esters or a pharmaceutically acceptable salt according to claim 1 comprising
a successive conversion of a propargyl alcohol in a propargyl ester by simple esterification,
a conversion of the propargyl ester in N-(4-amino-2-butynyl)ester by Mannich condensation to give a compound of formula I and, optionally, converting a compound of formula I to a corresponding pharmaceutical salt.

8. Method according to claim 7,
wherein the Mannich condensation is performed in the presence of paraformaldehyd, an acid catalyst, Cu-salts and a solvent.

9. Pharmaceutical composition for use in therapy, comprising a compound according to claim 1, and a pharmaceutically-acceptable carrier, adjuvant, vehicle and/or diluent.

10. Method of treating a cell proliferative disorder comprising
administering to a patient benefiting from such a treatment at least one M4-(N-substituted amino)-2-butynyl-1-ester represented by formula I, its bis-(2-butynyl)diester and/or a pharmaceutically acceptable salt thereof, 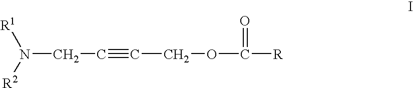
wherein
R is a hydrogen atom; a straight-chained or branched, saturated or unsaturated aliphatic radical with 1-20 C-atoms which is unsubstituted or substituted one or more times by C₁-C₆-alkyl, C₁-C₆-alkoxy, halogen, epoxy, amino, mercapto, a phenyl ring which is unsubstituted or substituted one or more times by C₁-C₆-alkyl, C₁-C₆-alkoxy, hydroxy, epoxy, amino, mercapto or halogen; a cycloalkyl group with 4 to 7 atoms unsubstituted or substituted one or more times by C₁-C₆-alkyl, C₁-C₆-alkoxy, hydroxy, epoxy, amino, mercapto or halogen,
R¹ and R² are joined to form a heterocyclic ring with 3 to 6 C-atoms, unsubstituted or substituted one or more times by C₁-C₆-alkyl, C₁-C₆-alkoxy, hydroxy, halogen, epoxy, amino, mercapto, whereby at least one C-atom can be replaced by 0, S or N, or
R¹ and R² are the same or different a hydrogen atom, a straight-chained or branched, saturated or unsaturated aliphatic radical with 1-20 C-atoms, unsubstituted or substituted one or more times by C₁-C₆-alkyl, C₁-C₆-alkoxy, hydroxy, halogen, epoxy, amino, mercapto,
in a cell proliferative disorder treating effective amount.

11. The method of claim 10, wherein the cell proliferative disorder is a neoplasia.

12. The method of claim 11, wherein the neoplasia is selected from the group consisting of leukemias, lymphomas, sarcomas, carcinomas, neural cell tumors, squamous cell carcinomas, germ cell tumors, undifferentiated tumors, seminomas, melanomas, neuroblastomas, mixed cell tumors, metastatic neoplasia and neoplasia due to pathogenic infections.

13. 4-(N-substituted amino)-2-butynyl-1-esters according to claim 5, wherein R is methyl, ethyl, propyl, butyl, pentyl, hexyl, phenyl, tertiary butyl or cyclohexyl.

14. Pharmaceutical composition for use in therapy, comprising a compound according to claim 3, and a pharmaceutically-acceptable carrier, adjuvant, vehicle and/or diluent.

15. A kit for inhibiting abnormal cell growth comprising at least one of the esters of claim 1, bis-(2-butynyl)diesters or pharmaceutically acceptable salts thereof, and, in a separate container, information about using parts of the kit.
